(19) United States
(12) Patent Application Publication
(10) Pub. No.: US 2022/0161075 A1
(43) Pub. Date: May 26, 2022

(54) Wildfire and Public Safety Event Detection Systems and Methods

(71) Applicant: GRID20/20, Inc., Richmond, VA (US)

(72) Inventors: W. Alan Snook, York, PA (US); Sergio Angeli, San Clemente, CA (US); Joseph O. Beaudet, Disputanta, VA (US)

(73) Assignee: GRID20/20, Inc., Richmond, VA (US)

(21) Appl. No.: 16/953,788

(22) Filed: Nov. 20, 2020

(51) Int. Cl. A62C 3/02; G01V 1/22; G01P 5/00; G01N 33/00; G08B 21/18; H04Q 9/00

(52) U.S. Cl. CPC A62C 3/0271; G01V 1/22; G01P 5/00; H04Q 2209/40; G08B 21/18; H04Q 9/00; G01N 33/0075

(57) ABSTRACT

A fire mitigation and/or public safety event detection device has a housing having a cavity and a plurality of varying sensors coupled within the cavity of the housing, and/or coupled to, or somehow connected or interfaced with the housing. The device and/or system further has a processor configured to determine, based upon outputs from the sensors, whether there is a fire/wildfire event or a public safety event or associated conditions. The processor is further configured to transmit data indicative of the sensors and alerts related to the fire event and/or the public safety event and/or associated conditions to one or more computing device(s) of utility personnel, authorized third parties, and/or first responders. An aggregated deployment of such fire mitigation and public safety event detection devices serves to create a web or network, thereby serving as a watchdog system within the associated deployment area(s).

### BACKGROUND

A wildfire is typically an unplanned, unwanted, and uncontrolled fire. The wildfire often starts in an area that contains combustible vegetation like a forest that has not been cleaned or thinned, so there is thick brush on the ground. A wildfire may also occur in populated areas where and when conditions are favorable (e.g., drought/dry period, low humidity, substantial winds, etc.) A wildfire may also be referred to as a forest fire, brush fire, bushfire, desert fire, grassfire, hill fire, peat fire, prairie fire, vegetation fire, or veld fire.

Many wildfires originate in rural areas. In rural areas, a wildfire can go unnoticed for hours or days. In such a scenario, the wildfire has already had time to grow in strength and destructiveness.

Wildfires can originate in different ways, e.g., lightning. When a forest is dry due to lack of rain it is easily ignited by lightning. Notably, other factors that contribute to wildfires include the earth's cover of carbon-rich vegetations, seasonally dry climates, atmospheric oxygen, and volcanic ignitions.

However, the most common cause of wildfires in some areas of the world is lightning. In other parts of the world human activity plays a role, e.g., arson, agriculture, animal husbandry, and land-conversion burning. In the United states many wildfires can be traced both to lightning strikes and human activities such as machinery sparks, cast-away cigarette butts, or arson.

Notably, severe wildfires occur when all the necessary elements of a fire come together in a susceptible area: an ignition source is brought into contact with a combustible material such as vegetation, that is subjected to enough heat and has an adequate or exceptional supply of oxygen from the ambient air, commonly associated with winds.

Typically, fire personnel become involved in trying to suppress a wildfire long after the wildfire has started. This delay in fighting the wildfires makes it extremely more difficult to get a wildfire under control.

Regardless of how the wildfire started, and the delay in fighting the wildfire, wildfires are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a wildfire occurs.

In addition to wildfires, a series of additional public safety events are also conceivable. For example, nuclear radiation emissions, noxious gas emissions, air quality conditions, seismic activity, etc. may also contribute to risk of human life, human injury, environmental damage, agricultural damage, economic impact, legal costs, suppression costs, etc.

Detection, early warning notification, and/or prevention are all imperative when it comes to responding to fires, wildfires, and/or public safety events. Time is of the essence when it comes to saving lives, reducing injuries, reducing damages, and reducing typical response costs.

Ongoing/perpetual monitoring provided by the fire mitigation and/or public safety event detection devices, when aggregated throughout a geographic area delivers a watchdog system for said area(s).

### DESCRIPTION OF THE DRAWINGS

The present disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1A is a depiction of an environment in which multiple exemplary fire mitigation and/or public safety event detection devices may be used for early detection and/or automated notification, and/or prevention to authorized parties of such events and/or associated conditions occurring.

FIG. 1B is a depiction of a rural environment in which multiple exemplary fire mitigation and/or public safety event detection devices may be used for early detection and/or automated notification to authorized parties of such events and/or associated conditions occurring.

FIG. 2 is an exemplary general form factor of a battery powered fire mitigation and/or public safety event detection device bearing onboard sensors as is shown in FIGS. 1 and 2.

FIG. 3 is an exemplary general form factor of a battery powered fire mitigation and/or public safety event detection device bearing onboard sensors as is shown in FIGS. 1 and 2 also having an external wired sensor pack.

FIG. 4 is an exemplary general form factor of a battery powered fire mitigation and/or public safety event detection device as is shown in FIGS. 1 and 2 bearing onboard sensors and having an externally coupled sensor pack attached thereto.

FIG. 5 is an exemplary general form factor of a battery powered fire mitigation and/or public safety event detection device as is shown in FIGS. 1 and 2 also having an externally coupled sensor pack attached thereto and an external wired sensor pack.

FIG. 6 is an exemplary general form factor fire mitigation device as is shown in FIGS. 1 and 2 having an external wired sensor pack and one or more voltage leads for powering the general form factor fire mitigation and/or public safety event detection device, and/or measuring voltages.

FIG. 7 is an exemplary general form factor fire mitigation device as is shown in FIGS. 1 and 2 having one or more voltage leads for powering the general form factor fire mitigation and/or public safety event detection device, and/or measuring voltages.

FIG. 8 is an exemplary general form factor fire mitigation and/or public safety event detection device as is shown in FIGS. 1 and 2 also having an externally coupled sensor pack attached thereto, and one or more voltage leads for powering the general form factor fire mitigation and/or public safety event detection device, and/or measuring voltages.

FIG. 9 is an exemplary general form factor of a fire mitigation and/or public safety event detection device as is shown in FIGS. 1 and 2 bearing onboard sensors, also having an externally coupled sensor pack attached thereto, one or more voltage leads for powering the general form factor of the fire mitigation and/or public safety event detection device and/or measuring voltages, and an external wired sensor pack.

FIG. 10 is a form factor of a battery powered fire mitigation and/or public safety event detection device as is shown in FIGS. 1 and 2, bearing onboard sensors,

FIG. 11 is a form factor of a battery powered fire mitigation and/or public safety event detection device as is shown in FIGS. 1 and 2, bearing onboard sensors, and further having an external wired sensor pack.

FIG. 12 is a form factor of a battery powered fire mitigation and/or public safety event detection device as is shown in FIGS. 1 and 2, bearing onboard sensors, and further having an externally coupled sensor pack attached thereto.

FIG. 13 is a form factor of a battery powered fire mitigation and/or public safety event detection as is shown in FIGS. 1 and 2, bearing onboard sensors, further having an externally coupled sensor pack attached thereto and an external wired sensor pack.

FIG. 14 is a form factor of a fire mitigation and/or public safety event detection device as is shown in FIGS. 1 and 2, bearing onboard sensors, and having one or more voltage leads for powering the form factor fire mitigation and/or public safety event detection device and/or measuring voltages.

FIG. 15 is a form factor of a fire mitigation and/or public safety event detection device as is shown in FIGS. 1 and 2, bearing onboard sensors, having one or more voltage leads for powering the form factor fire mitigation and/or public safety event detection device and/or measuring voltages, and an external wired sensor pack.

FIG. 16 is a form factor of a fire mitigation and/or public safety event detection device as is shown in FIGS. 1 and 2, bearing onboard sensors, having one or more voltage leads for powering the form factor fire mitigation and/or public safety event detection device and/or measuring voltages, and an externally coupled sensor pack attached thereto.

FIG. 17 is a form factor of a fire mitigation and/or public safety event detection device as is shown in FIGS. 1 and 2, bearing onboard sensors, having one or more voltage leads for powering the form factor fire mitigation and/or public safety event detection device and/or measuring voltages, an externally coupled sensor pack attached thereto, and an external wired sensor pack.

FIG. 18 is an exemplary battery powered standalone fire mitigation and/or public safety event detection device, and/or an external sensor pack for attaching to the fire mitigation and/or public safety event detection device as shown in FIGS. 1 and 2, bearing onboard sensors.

FIG. 19 is an exemplary standalone fire mitigation and/or public safety event detection device, and/or an external sensor pack for attaching to the fire mitigation and/or public safety event detection device as shown in FIGS. 1 and 2, bearing onboard sensors, and having one or more voltage leads for powering the stand alone, and/or external sensor pack, and/or for measuring voltages.

FIG. 20 is an exemplary battery powered standalone fire mitigation and/or public safety event detection device, and/or external sensor pack for attaching to the fire mitigation and/or public safety event detection device as shown in FIGS. 1 and 2, bearing onboard sensors, and having an external wired sensor pack attached thereto.

FIG. 21 is an exemplary standalone fire mitigation and/or public safety event detection device, and/or external sensor pack for attaching to the fire mitigation and/or public safety event detection device as shown in FIGS. 1 and 2, bearing onboard sensors, having one or more voltage leads for powering the standalone and/or external sensor pack and/or measuring voltages, and an external wired sensor pack.

FIG. 22 is an exemplary faceplate for a fire mitigation and/or public safety event detection device.

FIG. 23 is an exemplary sensor tray that comprises a substantially rectangular inner cavity.

### DETAILED DESCRIPTION

The present disclosure relates to fire mitigation and/or public safety event detection devices that may be used with or without an external power source. In populated and/or semi-populated areas where distribution transformers, other grid assets, and/or other power sources are prevalent the fire mitigation and/or public safety event detection devices can harvest power from the transformer, other grid assets, and/or other power sources. In more rural areas and/or wherever satisfactory power sources are unavailable, the fire mitigation and/or public safety event detection devices may have internal power sourcing, e.g., a battery.

In operation, the fire mitigation and/or public safety event detection devices comprise sensors, for example, sensors or probes for monitoring infrared imaging, monitoring vibration, ambient temperature, detecting seismic activity, measuring air quality, environmental wind direction and speed, host transformer exterior temperature, humidity, ground and/or surface temperature occurring below and/or nearby the respective deployment location(s), detecting the presence of smoke, the presence of nuclear radiation, the presence of noxious gases, circuit faults, and/or geo-positioning, etc.

If the fire mitigation and/or public safety event detection device detects one or more anomalous readings of the listed measurements, the device(s) and/or the associated software analyzes the data to determine whether a fire/wildfire and/or public safety event, and/or associated conditions is occurring. If a fire/wildfire and/or public safety event is or appears to be occurring, the fire mitigation and/or public safety event detection device(s) and/or associated software is configured to report such data to utility operators, authorized third parties, operating systems, and/or artificial intelligence platforms. Thus, action to fight the fire/wildfire and/or public safety event may be taken to ensure that the fire/wildfire and/or public safety event can be addressed readily, or more readily. Additionally, ongoing field-level data being reported by the fire/wildfire and/or public safety event detections devices can further assist response efforts during the respective event(s).

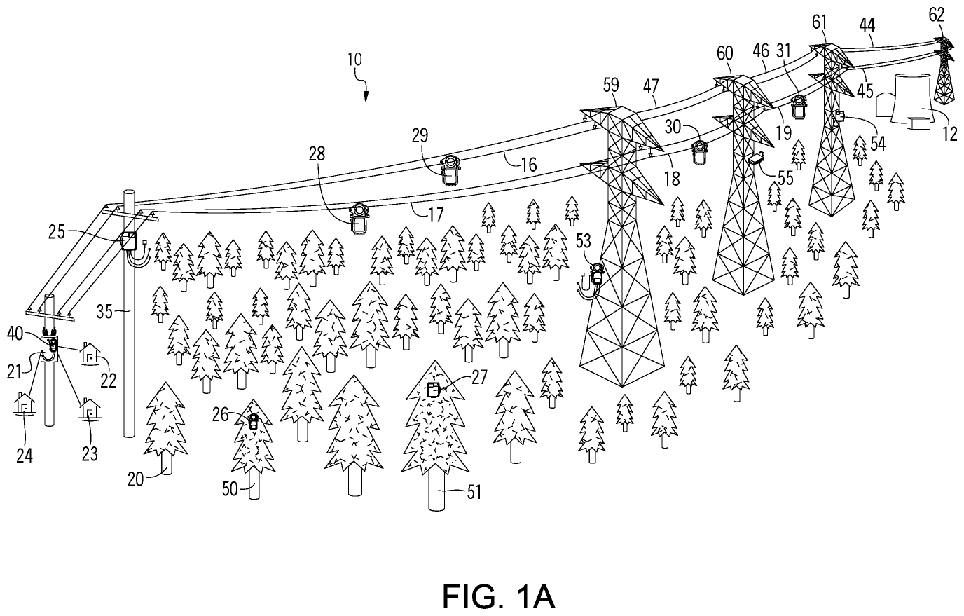

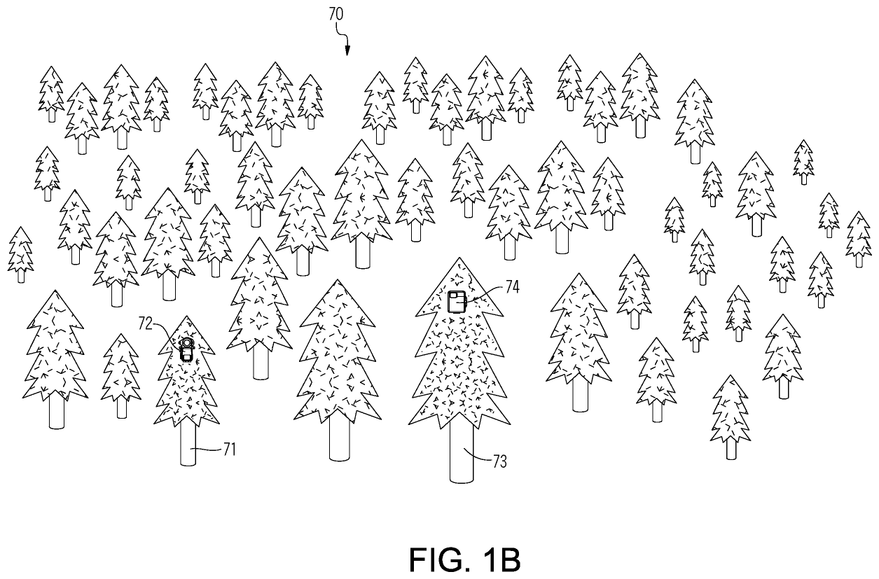

FIG. 1A depicts an environment 10 that is in a rural and semi-rural area. In this regard, there are homes 22-24 that are electrically coupled to a transformer 21. Note that only three houses 22-24 are shown and only one transformer 21 is shown; however, there may be more or fewer homes and more or fewer transformers in other embodiments.

Then environment comprises a power generation facility 12 and a plurality of electrical towers 59-62 that are coupled via power lines 44 and 45, 19 and 46, 18 and 47, and power lines 17 and 16 electrically couple to a power pole 35. In operation, the generation 12 provides power to transmissions lines 44, 45, 46, 47, 18, 19, 16, 17, as supported by towers 59-62, which transmits the power, ultimately downstream to a utility pole 35, and/or to and through a distribution substation (not shown). Conductors from the utility pole 35 supply power downstream which ultimately facilitate power to the homes 22-24.

Fire mitigation and/or public safety event detection devices of varying form factor(s) are placed like a network or a web throughout the environment 10. In this regard, a general form factor fire mitigation and/or public safety event detection device 40 is placed on the transformer 21. Additionally, general form factors of the fire mitigation and/or public safety event detection devices 28, 29, 30, and 31 may be coupled to transmission lines 44, 45, 46, 47, 17, 16, 18, and 19, and/or to downstream distribution lines that ultimately serve to deliver power to transformers and/or to downstream houses or other customer endpoints. Further, a general form factor fire mitigation and/or public safety event detection device such as 53 may be coupled to an electrical tower 56, and a general form factor fire mitigation and/or public safety event detection device such as 26 may be coupled to a tree 50.

Any variety of form factors may also be used throughout the deployed area. In this regard, a form factor fire mitigation and/or public safety event detection device such as 25 may be coupled to the electrical pole 35 and may harvest power from the electric grid and/or be battery powered. Further, a form factor fire mitigation and/or public safety event detection device 54 and 55 may be coupled to electrical towers 61, 60, respectively, and may harvest power from the electric grid, and/or be battery powered. In addition, a form factor of a fire mitigation and/or public safety event detection device such as 27 may be coupled to tree 51 and operate via battery power.

Note that general form factor fire mitigation and/or public safety event detection device such as 40 may derive power from the transformer 21, or be battery powered. The general form factor fire mitigation and/or public safety event detection devices such as 28, 29, 30, and 31 may derive power from the conductor cables 17, 16, 18, and 19, and/or be battery powered. General form factor fire mitigation and/or public safety event detection device such as 53 may derive power from the electrical grid attached to tower 56, and/or use battery power. However, general form factor fire mitigation and/or public safety event detection device such as 26 coupled to tree 50 has its own power supply, e.g., a battery perhaps recharged via a solar panel.

Also, form factor fire mitigation and/or public safety event detection devices 55 and 54 may derive power from the electrical towers 60 and 61, respectively, and/or be battery powered. The form factor fire mitigation and/or public safety event detection device such as 27 coupled to tree 51 may have an internal power source, e.g., a battery with or without a solar panel.

Regardless of the type of fire mitigation and/or public safety event detection device, each fire mitigation and/or public safety event detection device 40, 28, 29, 30, 31, 54, 55, 53, 26, and 27 all contain and/or are coupled to a plurality of sensors. The sensors may detect and monitor infrared imaging, monitoring vibration, ambient temperature, detecting seismic activity, measuring air quality, environmental wind direction and speed, transformer exterior temperature, humidity, ground and/or surface temperature occurring below and/or nearby the respective deployment location(s), detecting the presence of smoke, the presence of nuclear radiation, the presence of noxious gases circuit faults, and/or geo-positioning, etc.

If one or many fire mitigation and/or public safety event detection devices 40, 28, 29, 30, 31, 54, 55, 53, 27, and 26 receive data from its sensors that a fire/wildfire and/or public safety event condition is likely, the fire mitigation and/or public safety event detection devices 40, 28, 29, 30, 31, 54, 55, 53, 27, and 26 communicate an alert or a critical alert to a central computing device, to utility personnel, authorized third parties, first responders, authorized operating systems, artificial intelligence platforms and/or the like. Types of methods for backhauling this detected data to a central computing device, operations systems, artificial intelligence platforms, and/or other parties can be cellular, global system for mobile communications (GSM), radio frequency (RF) mesh, satellite, programmable logic controller, power line carrier (PLC), or any other means to backhaul the data to a central computing device (not shown).

Note that a computing device is broadly construed as any type of device capable of receiving messages from the fire mitigation and/or public safety event detection devices. For example, the computing device may be a handheld device like a cell phone, a tablet, a computer, or a laptop, etc.

Early detection and/or warning of a fire/wildfire and/or public safety event is beneficial. It allows first responders and/or authorized third parties to get to the scene quicker and/or to have access to data that can help guide and/or facilitate the appropriate response effort. Thus, the fire mitigation and/or public safety event detection devices 40, 28, 29, 30, 31, 54, 55, 53, 27, and 26 can help to save property, lives, the environment, and reduce legal and economic impacts.

Regardless of how the fire/wildfire and/or public safety event started, and the delay in responding, fires/wildfires and/or public safety events are a danger to human life, personal property, the environment, localized economies, wildlife, etc. Many human lives and/or injuries can be at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fire/wildfires, a series of additional public safety events are also conceivable. For example, nuclear radiation emissions, noxious gas emissions, air quality conditions, seismic activity, etc. may also contribute to the risk of human life, human injury, environmental damage, agricultural damage, economic impact, legal costs, suppressions costs, etc.

FIG. 1B depicts a rural area 70, e.g., a forest. In a forest, there may be no source of power to operate fire mitigation and/or public safety event detection devices. The rural area in FIG. 1B comprises a general form factor fire mitigation and/or public safety event detection device 72 that is coupled to a tree 71. Further, the rural area comprises a separate fire mitigation and/or public safety event detection device 74 that is coupled to a tree 73. Note that only two fire mitigation and/or public safety event detection devices are shown; however, many more fire mitigation and/or public safety event detection devices may be installed in the environment 70 to create a network or web for detection, and/or warning of fires/wildfires and/or public safety events.

Note that the fire mitigation and/or public safety event detection devices 72 and 74 are not near a power source. Therefore, each fire mitigation and/or public safety event detection device 72 and 74 comprise an internal power source, e.g., a battery potentially with or without a solar panel.

As noted above, the fire mitigation and/or public safety event detection devices 72 and 74 comprise a plurality of onboard and/or external sensors. In this regard, the sensors may detect and monitor infrared imaging, monitoring vibration, ambient temperature, detecting seismic activity, measuring air quality, environmental wind direction and speed, transformer exterior temperature, humidity, ground and/or surface temperature occurring below and/or nearby the respective deployment location(s), detecting the presence of smoke, the presence of nuclear radiation, the presence of noxious gases and/or geo-positioning, etc.

If one or many fire mitigation and/or public safety event detection devices 72 and 74 receive data from its associated sensors indicating that a fire/wildfire and/or public safety event is likely or is occurring, the fire mitigation and/or public safety event detection devices 72 and 74 communicate an alert or a critical alert to a central computing system, operations systems, artificial intelligence platforms and/or authorized third parties such as but not limited to first responders, etc. Types of methods for backhauling this detected data to a central computing device and/or third parties can be cellular, global system for mobile communications (GSM), radio frequency (RF) mesh, satellite, programmable logic controller, power line carrier (PLC), or any other means to backhaul the data to the central computing device.

Early warning of a fire/wildfire and/or public safety event is beneficial. It allows first responders and/or authorized third parties to respond more promptly, and/or to receive and possibly maintain a source of field-derived data flow throughout the duration of such event(s). Thus, the fire mitigation and/or public safety event detection devices 72 and 74 can save property, lives, injuries, the environment, wildlife, suppression/remediation costs, economic impact, legal costs, liability costs, etc.

Regardless of how the fire/wildfire and/or public safety event started, and the duration of delay in responding, fires/wildfires and/or public safety events are a danger to human life, personal property, the environment, localized economies, wildlife, financial stability of utilities, localized economies, and insurers, etc. Many human lives and/or injuries can be at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, seismic activity, etc. may also contribute to risk of human life, human injury, environmental damage, agricultural damage, economic impact, legal costs, suppressions costs, etc.

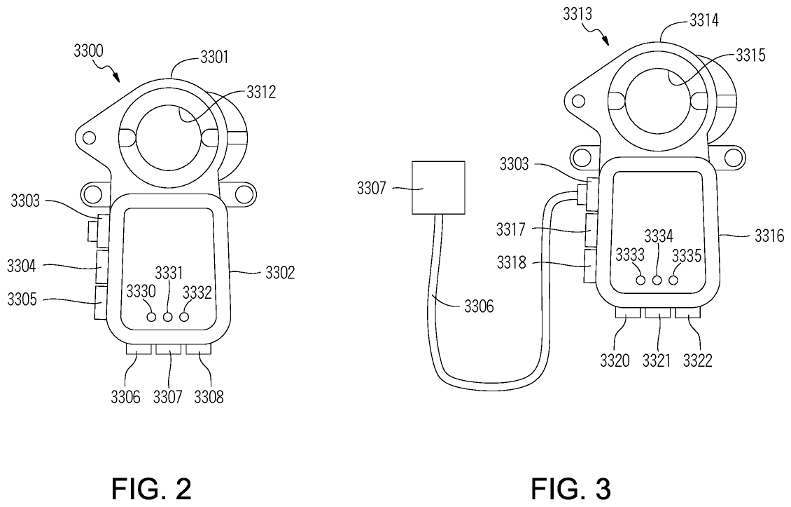

FIG. 2 is a general form factor of a fire mitigation and/or public safety event detection device 3300 bearing on board sensors. The general form factor fire mitigation and/or public safety event detection device 3300 comprises a head unit 3301 and an extended box-shaped unit 3302 integral with the head unit 3301.

The general form factor fire mitigation and/or public safety event detection device 3300 comprises a plurality of ports 3303-3305 that extend laterally from a side of the extended box-shaped unit. Other sensors (not shown), other devices (not shown) or power conductors (not shown) may be coupled to the plurality of ports 3303-3305.

Also, the general form factor fire mitigation and/or public safety event detection device 3300 comprises a plurality of ports 3306-3308 that extend laterally from a bottom of the extended box-shaped unit. Other sensors (not shown), other devices (not shown) or power conductors (not shown) may be coupled to the plurality of ports 3306-3308.

Also, the head unit 3301 comprises an opening 3312. The opening 3312 may help in mounting the general form factor fire mitigation and/or public safety event detection device 3300 to an object. For example, when mounting to a tree, the opening 3312 may go around a branch, or when mounting on a power line the opening 3312 may go around a power line, etc.

The general form factor fire mitigation and/or public safety event detection device 3300 also comprises two light-emitting diodes (LED) 3309 and 3310. The LEDs 3309 and 3310 can communicate the status of the general form factor fire mitigation and/or public safety event detection device 3300. For example, if the lights are green, that may indicate that the general form factor fire mitigation and/or public safety event detection device 3300 is operating properly. However, if the lights are red, that may indicate that the general form factor fire mitigation and/or public safety event detection device 3300 is not operating properly.

Further, the general form factor fire mitigation and/or public safety event detection device 3300 comprises an optical port 3311. The optical port 3311 connects with other sensors (not shown) and other devices (not shown) wired or wirelessly.

Within the extended box-shaped unit is a plurality of onboard sensors. The sensors may collect and monitor data indicative of infrared imaging, vibration, ambient temperature, detecting seismic activity, air quality, environmental wind direction and speed, humidity, ground and/or surface temperature occurring below and/or nearby the respective deployment location(s), the presence of smoke, the presence of nuclear radiation, the presence of noxious gases and/or geo-positioning, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

If the general form factor fire mitigation and/or public safety event detection device 3300 receives data from its sensors that a fire/wildfire and/or public safety event is likely, the general form factor fire mitigation and/or public safety event detection device 3300 communicates an alert or a critical alert to a central computing system, and/or to utility personnel, authorized third parties, first responders, or the like. Types of methods for backhauling this detected data to a central computing device and/or other entities can be cellular, global system for mobile communications (GSM), radio frequency (RF) mesh, satellite, programmable logic controller, power line carrier (PLC), or any other means to backhaul the data to the central computing device.

Early warning of a fire/wildfire and/or public safety event is beneficial. It allows first responders and authorized third parties to respond more quickly. Thus, the general form factor fire mitigation and/or public safety event detection device 3300 can save property, the environment, wildlife, human injury, and lives.

Regardless of how the fire/wildfire and/or public safety event started, and the delay in responding, fires/wildfires and public safety events are a danger to human life, personal property, the environment, localized economies, wildlife, etc. Many human lives are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

The general form factor fire mitigation and/or public safety event detection device 3300 does not have access to an external power source. Therefore, the general form factor fire mitigation and/or public safety event detection device 3300 may use solar energy or a battery or both to harvest power for operation.

Also, the head unit 3301 comprises an opening 3312. The opening may help in mounting the general form factor fire mitigation and/or public safety event detection device 3300 to an object. For example, when mounting to a tree, the opening 3306 may go around a branch, or when mounting on a power line, the opening 3306 may go around a power line, etc.

FIG. 3 is a general form factor fire mitigation and/or public safety event detection device bearing onboard sensors 3313 in accordance with an embodiment of the present disclosure. The general form factor fire mitigation and/or public safety event detection device 3313 is like the general form factor fire mitigation and/or public safety event detection device 3300 (FIG. 2). In this regard, the general form factor fire mitigation and/or public safety event detection device 3313 comprises all the functionality of the general form factor fire mitigation and/or public safety event detection device 3300.

The general form factor fire mitigation and/or public safety event detection device 3313 comprises a head unit 3314 and an extended box-shaped unit 3316. Within the box-shaped unit 3316 resides one or more printed circuit boards (PCB) (not shown), semiconductor chips (not shown), and/or other electronics and/or onboard sensors (not shown) for performing operations related to the general form factor fire mitigation and/or public safety event detection device 3313. In one embodiment, the housing section box-shaped unit 3316 is a substantially rectangular housing; however, differently sized, and differently shaped housings may be used in other embodiments.

Also, the head unit 3314 comprises an opening 3315. The opening may help in mounting the general form factor fire mitigation and/or public safety event detection device 3300 to an object. For example, when mounting to a tree, the opening 3306 may go around a branch, or when mounting on a power line, the opening 3306 may go around a power line, etc.

Like the general form factor fire mitigation and/or public safety event detection device 3300, the box-shaped unit 3316 is not powered by an external source. Instead, the box-shaped unit 3316 comprises a battery (not shown) that powers the box-shaped unit 3316. Solar power may also be used in conjunction with the battery.

Within the box-shaped unit 3316 are a plurality of onboard sensors. The sensors may include, but are not limited to, an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, transformer can temperatures, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

The general form factor fire mitigation and/or public safety event detection device 3313 further comprises ports 3317-3318. Other sensors may be tethered to these ports or wirelessly connected to these ports. The sensors may collect data regarding the surrounding environment, and/or an associated nearby or host assets upon which the general form factor fire mitigation and/or public safety event detection device 3313 is mounted.

Also, the general form factor fire mitigation and/or public safety event detection device 3313 comprises a plurality of ports 3320-3322 that extend laterally from a bottom of the extended box-shaped unit 3316. Other sensors (not shown), other devices (not shown) or power conductors (not shown) may be coupled to the plurality of ports 3320-3322.

Dissimilar to the general form factor fire mitigation and/or public safety event detection device 3300, the general form factor fire mitigation and/or public safety event detection device 3313 also comprises an external wired sensor pack 3307. The external wired sensor pack 3307 is communicatively coupled via cable 3306 and port 3303 to the general form factor fire mitigation and/or public safety event detection device 3313. The external wired sensor pack 3307 may measure, for example, other data necessary for determining if a fire/wildfire and/or public safety event is present.

In operation, the general form factor fire mitigation and/or public safety event detection device 3313 receives power from a battery (not shown). The battery power operates the various sensors described above. Note that solar power may be used as well.

The sensors continuously gather information about the environment to determine if a public safety event and/or fire(s)/wildfire(s) conditions and/or activities, and/or useful event prevention data are detected.

In this regard, the infrared imaging camera(s) can obtain images that indicate a fire/wildfire and/or public safety event or related conditions. A smoke sensor can sense the presence of smoke, and/or noxious gases, etc. Other sensors may detect the presence of nuclear radiation, seismic activity, humidity change, etc. If the general form factor fire mitigation and/or public safety event detection device 3313 detects a public safety event and/or fire/wildfire, the general form factor fire mitigation and/or public safety event detection device 3313 transmits data to a monitoring device, which can be a computer, an operations system(s), etc. thereby indicating that there is a public safety event and/or fire/wildfire condition(s) present. The associated computer and/or operations system(s), etc. may be manned by utility personnel, first responders, or the like.

The data indicating a public safety event and/or fire/wildfire is present and/or conditions infer such an event may be transmitted in any number of communication methods. In this regard, the communication to the computer and/or operations system(s), etc. may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to utility personnel, authorized third parties, and/or first responders.

Note that the general form factor fire mitigation and/or public safety event detection device 3313 may backhaul data, including data indicative of smoke and/or noxious gas sensors, infrared camera images, ambient and/or surface temperatures, humidity, nuclear radiation, seismic activity, weather, etc.

Regardless of where the data originates, the general form factor fire mitigation and/or public safety event detection device 3313 backhauls the data. The general form factor fire mitigation and/or public safety event detection device 3313 transmits the data to personnel, including utility personnel, authorized third parties, and/or first responders, and/or to operations systems.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a wildfire breaks out.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

In addition, the general form factor fire mitigation and/or public safety event detection device 3313 may comprise an antenna (not shown). The antenna shall allow improved communications to the central computing device(s), operations systems, artificial intelligence platforms, authorized third parties, utilities, first responders, etc.

In one embodiment, the general form factor fire mitigation and/or public safety event detection device 3313 may comprise a magnet or bracket (not shown) on the back of the general form factor fire mitigation and/or public safety event detection device 3313. The magnet or bracket allow the general form factor fire mitigation and/or public safety event detection device 3313 to be attached to a transformer, a power line, an electrical tower, a power pole, a tree, a building, etc.

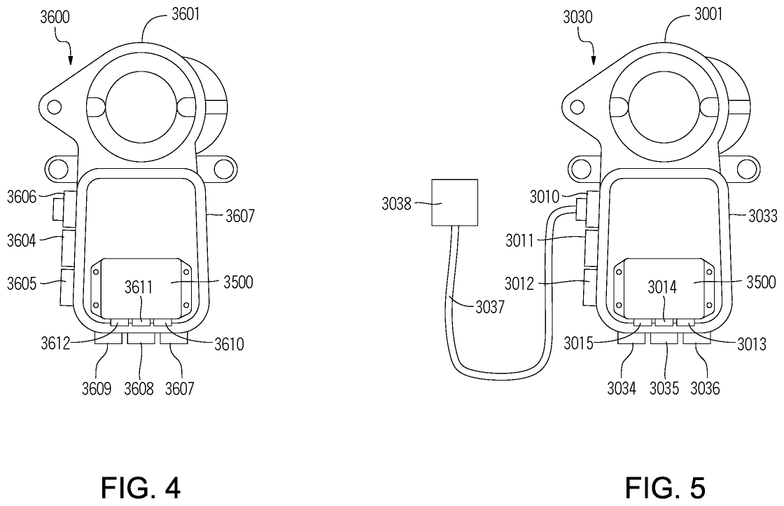

FIG. 4 is a general form factor fire mitigation and/or public safety event detection device 3600 in accordance with an embodiment of the present disclosure. The general form factor fire mitigation and/or public safety event detection device 3600 comprises an extended box-shaped unit 3607 that is integral with a head unit 3601.

The general form factor fire mitigation and/or public safety event detection device 3600 is powered by a battery source which may or may not include solar power.

The extended box-shaped unit 3607 may comprise a plurality of onboard sensors within the extended box-shaped unit 3607. The sensors contained in the extended box-shaped unit 3607 may comprise, but is not limited to, an infrared imaging camera(s), vibration sensors, ambient temperature sensors (e.g., approximately 30-feet high where transformers reside), transformer can temperature, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Additionally, the externally coupled sensor pack 3500 may also contain a plurality of sensors including, but not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, and/or fault sensors, geo-positioning sensors, etc. The externally coupled sensor pack may contain one (1) or more power cables (not shown) to operate the sensor pack 3500, and/or the externally coupled sensor pack 3500 can be powered via battery which may or may not include solar, and/or via power harvesting from the host device 3601.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

Further, the externally coupled sensor pack comprises a plurality of ports 3610-36-12. The ports 3610-3612 may be connected to power sources (not shown), sensors (not shown) or another device (not shown).

Further, the general form factor fire mitigation and/or public safety event detection device 3600 comprises a plurality of ports 3604-3606 and 3609-3607. The ports 3604-3606 and 3609-3607 may be connected to power sources (not shown), sensors (not shown) or another device(s) (not shown).

In operation, the general form factor fire mitigation and/or public safety event detection device 3600 may collect data that indicates that a public safety event and/or fire/wildfire is present and/or conditions similar to such events are present thereby providing event awareness/early awareness and/or prevention opportunity for operators, first responders, authorized third parties, etc., and/or to support artificial intelligence platforms for predicting emerging and/or imminent issues of interest or concern. Notably, the fire mitigation and/or public safety event detection device 3600 conjoins data obtained from the onboard sensors in the extended box-shaped unit 3607 and/or data obtained from the sensors in the externally coupled sensor pack 3500, Notably, the externally coupled sensor pack 3500 is communicatively coupled to the electronics contained in the extended box-shaped unit 3500.

Further, the sensors continuously gather information about the localized surroundings to determine if public safety and/or fire(s)/wildfire(s) conditions and/or activities are detected, and/or to detect useful event prevention data, and/or to facilitate artificial intelligence. In this regard, the infrared imaging camera(s) can obtain data that indicate a potential or active fire/wildfire and/or public safety event. The smoke sensor can sense the presence of smoke and/or noxious gas, etc. Additional sensors associated with the fire mitigation and/or public safety event detection device may also report revealing and/or useful data. If the general form factor fire mitigation and/or public safety event detection device 3600 detects a public safety event, and/or fire/wildfire conditions and/or activity, the fire mitigation and/or public safety event detection device may transmit data to a monitoring device (not shown), or platform(s) which can be a computer, operations system, phone, etc., thereby indicating that a public safety event and/or fire/wildfire condition and/or activity is present.

The data indicating that a public safety event and/or fire/wildfire condition and/or activity is present may be transmitted in any number of communication methods. In this regard, the communication to the computer, platform, or operations system, etc. may be cellular, satellite, power line carrier (PLC), and/or radio frequency (RF) mesh. These are merely examples, and other methods may be used in other embodiments.

The public safety event and/or fire/wildfire condition or activity data transmitted may be in the form of an alert or a critical alert. Further, the alerts may be communicated to utility personnel, other authorized parties, and/or first responders, etc. Localized conditions may be transmitted to utility personal, or other authorized authorities to provide event prevention data.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to wildfires, a series of additional public safety events are also conceivable. For example, nuclear radiation emissions, noxious gas emissions, air quality conditions, seismic activity, etc. may also contribute to risk of human life, human injury, environmental damage, agricultural damage, economic impact, legal costs, suppressions costs, etc.

Note that the general form factor fire mitigation and/or public safety event detection device 3600 may backhaul data, including data indicative of smoke and/or noxious gas sensors, infrared camera images, ambient and/or surface temperatures, humidity, nuclear radiation, seismic vibration, weather, etc.

Regardless of where the data originates, the general form factor fire mitigation and/or public safety event detection device 3600 backhauls the data. The general form factor fire mitigation and/or public safety event detection device 3600 transmits the data to authorized personnel including but not limited to utility personnel and first responders, and/or to operations systems.

In addition, the general form factor fire mitigation and/or public safety event detection device 3600 may comprise an antenna (not shown). The antenna shall improve communications to including but not limited utilities, authorized third parties, and/or first responders, etc.

In one embodiment, the general form factor fire mitigation and/or public safety event detection device 3600 may comprise a magnet or bracket (not shown) on the back of the general form factor fire mitigation and/or public safety event detection device 3600. The magnet or bracket allow the general form factor fire mitigation and/or public safety event detection device 3600 to be attached to a transformer, a power pole, a transmission tower, a tree, a building, etc.

Furthermore, the general form factor fire mitigation and/or public safety event detection device 3600 is configured to be tethered to and/or wirelessly in communication with other sensors.

FIG. 5 is a general form factor fire mitigation and/or public safety event detection device 3030 in accordance with an embodiment of the present disclosure. The general form factor fire mitigation and/or public safety event detection device 3030 is like the fire mitigation and/or public safety event detection device 3300 (FIG. 2). In this regard, the general form factor fire mitigation and/or public safety event detection device 3300 comprises all the functionality of the fire mitigation and/or public safety event detection device 3030 except the general form factor fire mitigation and/or public safety event detection device 3030 comprises a coupled external sensor pack 3014 and an external wired sensor pack 3038.

The fire mitigation and/or public safety event detection device 3030 comprises an extended box-shaped unit 3033 bearing onboard sensors. Within the extended box-shaped unit 3033 resides one or more printed circuit boards (PCB) (not shown), semiconductor chips (not shown), and/or other electronics and/or sensors (not shown) for performing operations related to the fire mitigation and/or public safety event detection device 3300. In one embodiment, the extended box-shaped unit 3033 is a substantially rectangular housing; however, differently sized, and differently shaped housings may be used in other embodiments.

Within the extended box-shaped unit 3033 are a plurality of sensors. The sensors may include, but are not limited to, an infrared imaging camera(s), vibration sensors, ambient temperature sensors, transformer can temperature, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors, etc. The externally coupled sensor pack may contain one (1) or more power cables (not shown) to operate the sensor pack 3500, and/or the externally coupled sensor pack 3500 can be powered via battery which may or may not include solar, and/or via power harvesting from the host device 3001.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

The general form factor fire mitigation and/or public safety event detection device 3030 further comprises ports 3034-3036 and 3011 and 3012. Other sensors may be tethered to these ports or wirelessly connected to these ports. The sensors may collect data regarding the localized surrounding environment, and/or the host transformer associated with the general form factor fire mitigation and/or public safety event detection device 3030.

Further, the general form factor fire mitigation and/or public safety event detection device 3030 differs from fire mitigation and/or public safety event detection device 3300. In this regard, the general form factor fire mitigation and/or public safety event detection device 3030 comprises a port 3010 to which an external wired sensor pack 3038 may be coupled via cable 3037. The sensor pack may gather, for example, the can temperature of a transformer, and/or other sensor data points.

In operation, the general form factor fire mitigation and/or public safety event detection device 3030 receives power from a battery (not shown) which may or may not include solar power. The battery power operates the various sensors described above.

Further, the sensors continuously gather information about the localized surroundings, and/or the host transformer, or other host asset(s) to determine if a public safety event and/or fire(s)/wildfire(s) conditions and/or activities, and/or useful event prevention data are detected, and/or to facilitate ongoing localized surroundings and/or host asset(s) monitoring.

In this regard, the infrared imaging camera(s) can obtain images that indicate a fire/wildfire and/or public safety events and/or conditions. The smoke sensor can sense the presence of smoke, and/or noxious gases, etc. Other sensors may detect the presence of nuclear radiation, seismic activity, humidity change, etc. If the general form factor fire mitigation and/or public safety event detection device 3030 detects a public safety event and/or fire/wildfire, the fire mitigation and/or public safety event detection device may transmit data to a monitoring device, which can be a computer, authorized third parties, operations systems, and/or artificial intelligence indicating that there is a public safety event and/or fire/wildfire present and/or conditions associated with such events may be present.

Additionally, an externally coupled sensor pack 3500 may also contain a plurality of sensors including, but not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, and/or fault sensors, geo-positioning sensors, etc. The sensor pack 3500 is communicatively coupled to the general form factor fire mitigation and/or public safety event detection device 3030.

The external sensor pack 3500 comprises three ports 3013-3015. Other sensors may be tethered to these ports or wirelessly connected to these ports. The sensors may collect data regarding the surrounding environment, and/or the host transformer and/or other host asset(s) associated with the general form factor fire mitigation and/or public safety event detection device 3030.

The data indicating a public safety event and/or fire/wildfire is present may be transmitted in any number of communication methods. In this regard, the communication to the computer, authorized third parties, first responders, operations systems, etc. may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to utility personnel, authorized third parties, first responders, operations systems, artificial intelligence platforms, etc.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the general form factor fire mitigation and/or public safety event detection device 3030 may backhaul data, including data indicative of smoke and/or noxious gas sensors, infrared camera images, ambient and/or surface temperatures, humidity, nuclear radiation, seismic activity, host transformer can temperature, weather, etc.

Regardless of where the data originates, the fire mitigation and/or public safety event detection device 3030 backhauls the data. The general form factor fire mitigation and/or public safety event detection device 3030 transmits the data to personnel, including utility personnel, authorized third parties, and/or first responders, and/or to operations systems, and/or to artificial intelligence platforms.

In addition, the general form factor fire mitigation and/or public safety event detection device 3030 may comprise an antenna (not shown). The antenna shall allow improved communications to the central computing device(s), operations systems, artificial intelligence platforms, authorized third parties, utilities, first responders, etc.

In one embodiment, the general form factor fire mitigation and/or public safety event detection device 3030 may comprise a magnet or bracket (not shown) on the back of the general form factor fire mitigation and/or public safety event detection device 3030. The magnet or bracket allow the general form factor fire mitigation and/or public safety event detection device 3030 to be attached to an object like a power pole, a transformer, a tree, a building, and the like.

Furthermore, the general form factor fire mitigation and/or public safety event detection device 3030 is configured to be tethered to or wirelessly in communication with sensors that communicate with the fire mitigation and/or public safety event detection device 3030.

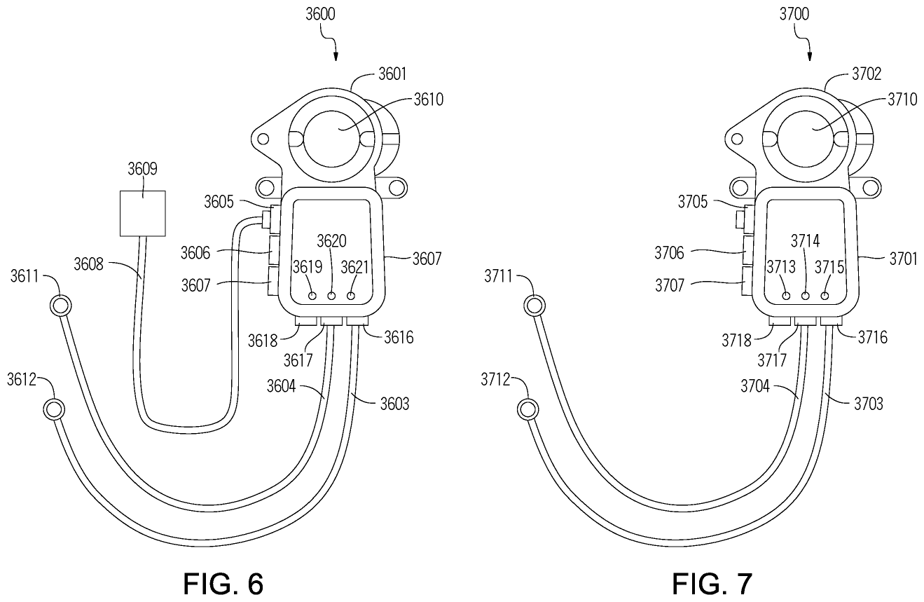

FIG. 6 is a general form factor fire mitigation and/or public safety event detection device 3600 in accordance with an embodiment of the present disclosure. The general form factor fire mitigation and/or public safety event detection device 3600 is like the general form factor fire mitigation and/or public safety event detection device 3300 (FIG. 2). In this regard, the general form factor fire mitigation and/or public safety event detection device 3600 comprises all the functionality of the fire mitigation and/or public safety event detection device 3300.

The general form factor fire mitigation and/or public safety event detection device 3600 comprises an extended box-shaped unit 3607 and a head 3601 integral with the extended box-shaped unit 3607 bearing onboard sensors. Within the extended box-shaped unit 3607 resides one or more printed circuit boards (PCB) (not shown), semiconductor chips (not shown), one or more onboard sensors (not shown), and/or other electronics and/or sensors (not shown) for performing operations related to the general form factor fire mitigation and/or public safety event detection device 3600. In one embodiment, the extended box-shaped unit 3607 is a substantially rectangular housing; however, differently sized, and differently shaped housings may be used in other embodiments.

Also, a head unit 3601 comprises an opening 3610. The opening 3610 may help in mounting the general form factor fire mitigation and/or public safety event detection device 3600 to an object. For example, when mounting to a tree, the opening 3610 may go around a branch, when mounting to a power line the opening 3610 may go around the power line, etc.

Within the extended box-shaped unit 3607 can be a plurality of onboard sensors. The sensors may include, but are not limited to, an infrared imaging camera(s), vibration sensors, ambient temperature sensors, transformer can temperature, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, and/or fault sensors, geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

The fire mitigation and/or public safety event detection device 3600 further comprises ports 3605, 3608 and 3607. Other sensors may be tethered to these ports and/or wirelessly connected to these ports. The sensors may collect data regarding the surrounding environment, the host transformer and/or the host assets associated with the general form factor fire mitigation and/or public safety event detection device 3600.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

On the extended box-shaped unit 3607 there are light-emitting diodes 3619 and 3620. The LEDs may light up different colors to communicate the status of the general form factor fire mitigation and/or public safety event detection device 3600. For example, if the general form factor fire mitigation and/or public safety event detection device 3600 is operating properly, the lights may be green; however, if there is a problem with the general form factor fire mitigation and/or public safety event detection device 3600, the lights may be red.

Further, the general form factor fire mitigation and/or public safety event detection device 3600 comprises an optical port 3621. The optical port 3621 connects with other sensors (not shown) and other devices (not shown), wired or wirelessly.

The general form factor fire mitigation and/or public safety event detection device 3600 also comprises ports 3605-3607 and 3616-3618. Sensors and/or power supplies may be coupled to these port in operation. The general form factor 3600 also bears an external wired sensor pack 3609, connected via cable 3608.

In operation, the general form factor fire mitigation and/or public safety event detection device 3600 receives power from an external source, e.g., a transformer (not shown), power line (not shown), etc. to which the fire mitigation and/or public safety event detection device 3600 is coupled using power source cables and/or voltage leads 3603 and 3604. The power source cables and/or voltage leads may be connected to the host power source using connectors 3611 and 3612, or other embodiments not shown. The power operates the various sensors described above.

Further, the sensors continuously gather information about the surrounding localized area, the host transformer, host asset(s), etc. to determine if a public safety event and/or fire(s)/wildfire(s) conditions and/or activities, and/or useful event prevention data are detected, and/or to facilitate ongoing asset and localized conditions monitoring, and/or support artificial intelligence platforms.

In this regard, the infrared imaging camera(s) can obtain images that indicate a public safety event and/or a fire/wildfire, or similar conditions. The smoke sensor can sense the presence of smoke, noxious gases, etc. If the general form factor fire mitigation and/or public safety event detection device 3600 detects voltage spikes and/or drops at the host transformer and/or host asset location which are indicative of downed, broken and/or faulty conductors, and/or fire/wildfire or public safety event, the fire mitigation and/or public safety event detection device 3600 may transmit data to a monitoring device, which can be a computer, authorized third parties, artificial intelligence platforms, etc. indicating that there is a public safety event and/or fire/wildfire present and/or conditions indicative of same are present.

The data indicating a fire/wildfire and/or public safety event or related conditions is present may be transmitted in any number of communication methods. In this regard, the communication to the computer may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to utility personnel, authorized third parties, first responders, operations systems, artificial intelligence platforms, etc.

Note that the general form factor fire mitigation and/or public safety event detection device 3600 may backhaul data, including data indicative of smoke and/or noxious gas sensors, infrared camera images, ambient and/or surface temperatures, humidity, nuclear radiation, seismic activity, voltage spikes or drops indicative of downed, broken and/or faulty conductors, weather, host transformer or host asset external temperature(s), etc.

Regardless of where the data originates, the fire mitigation and/or public safety event detection device 3600 backhauls the data. The general form factor fire mitigation and/or public safety event detection device 3600 transmits the data to central computing systems, and/or to personnel, including utility personnel, authorized third parties, and/or first responders, and/or to operations systems and/or to artificial intelligence platforms, etc.

In addition, the general form factor general form factor fire mitigation and/or public safety event detection device 3600 may comprise an antenna (not shown). The antenna shall allow improved communications to the central computing device(s), operations systems, artificial intelligence platforms, authorized third parties, utilities, first responders, etc.

In one embodiment, the general form factor fire mitigation and/or public safety event detection device 3600 may comprise a magnet or bracket (not shown) on the back of the general form factor fire mitigation and/or public safety event detection device 3600. The magnet or bracket allow the general form factor fire mitigation and/or public safety event detection device 3600 to be attached to the host transformer, host asset, tree, building, etc.

Furthermore, the general form factor fire mitigation and/or public safety event detection device 3600 is configured to be tethered to and/or wirelessly in communication with sensors that communicate with the general form factor fire mitigation and/or public safety event detection device 3600.

FIG. 7 is a general form factor fire mitigation and/or public safety event detection device 3700 bearing onboard sensors in accordance with an embodiment of the present disclosure. The general form factor fire mitigation and/or public safety event detection device 3700 is like the general form factor fire mitigation and/or public safety event detection device 3300 (FIG. 2). In this regard, the general form factor fire mitigation and/or public safety event detection device 3700 comprises all the functionality of the general form factor fire mitigation and/or public safety event detection device 3300. However, the general form factor fire mitigation and/or public safety event detection device 3700 also comprises power conductors 3703 and 3704 coupled to ports 3716 and 3717, by cables 3703 and 3704, which are described further herein.

The general form factor fire mitigation and/or public safety event detection device 3700 comprises an extended box-shaped unit 3701. Within the extended box-shaped unit 3701 resides one or more printed circuit boards (PCB) (not shown), semiconductor chips (not shown), and/or other electronics and/or sensors (not shown) for performing operations related to the general form factor fire mitigation and/or public safety event detection device 3000. In one embodiment, the extended box-shaped unit 3701 is a substantially rectangular housing; however, differently sized, and differently shaped housings may be used in other embodiments.

Additionally, the general form factor fire mitigation and/or public safety event detection device 3700 further comprises one or more cables 3703, 3704. The cables 3704, 3703 on device 3700 may be coupled to a conductor cable or corresponding bus bars or bushings (not shown) and ground or reference voltage conductor(s) (not shown), respectively, for the corresponding conductor cable with terminators 3711, 3712, respectively. The cables 3704, 3703 provide power to the fire mitigation and/or public safety event detection device 3700. Note that the conductor cables 3703, 3704 may be coupled to any type external power source.

On the extended box-shaped unit 33701 there are light-emitting diodes 3713 and 3714. The LEDs may light up different colors to communicate the status of the general form factor fire mitigation and/or public safety event detection device 3700. For example, if the general form factor fire mitigation and/or public safety event detection device 3700 is operating properly, the lights may be green; however, if there is a problem with the general form factor fire mitigation and/or public safety event detection device 3700, the lights may be red.

Further, the general form factor fire mitigation and/or public safety event detection device 3700 comprises an optical port 3715. The optical port 3715 connects in a wired and/or wireless manner with other sensors (not shown) and other devices (not shown).

Within the extended box-shaped unit 3701 are a plurality of onboard sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, voltage sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

Further, other sensors may be tethered to the general form factor fire mitigation and/or public safety event detection device 3700. In this regard, the fire mitigation and/or public safety event detection device 3700 comprises ports 3105, 3106, 3107, and 3718 to which other sensors may be connected.

In operation, the general form factor fire mitigation and/or public safety event detection device 3700 may receive power from a host transformer (not shown), or a host asset (not shown) from voltage leads also serving as power cables 3703 and 3704 to which the general form factor fire mitigation and/or public safety event detection device 3700 is coupled. The power may operate the various sensors described above.

Further, the sensors continuously gather information about the localized surrounding areas, the host transformer, and/or the host asset(s) to determine if a public safety event and/or fire(s)/wildfire(s) conditions and/or activities are occurring, and/or to provide useful event prevention data, and/or to facilitate ongoing host asset(s) and/or localized surrounding conditions monitoring, and/or to supply artificial intelligence platforms with useful data.

In this regard, the infrared imaging camera(s) can obtain images that indicate a fire/wildfire and/or public safety event. The smoke sensor can sense the presence of smoke, noxious gases, etc. If the fire mitigation and/or public safety event detection device 3700 detects fire/wildfire and/or a public safety event including but not limited to a broken, downed or faulty conductor, nuclear radiation, seismic activity, smoke detection, unusual ambient and/or surface temperature, etc., the fire mitigation and/or public safety event detection device 3700 may transmit data to a monitoring device, which can be a computer, operating system, artificial intelligence platform, etc. indicating that there is a public safety event and/or fire/wildfire present and/or related conditions have been detected.

The data indicating a public safety and/or fire/wildfire is present or similar conditions are detected may be transmitted in any number of communication methods. In this regard, the communication to the computer may be cellular, satellite, power line carrier (PLC), or radio frequency (RF) mesh. These are merely examples, and other methods may be used in other embodiments.

Regardless of how the fire/wildfire and/or public safety event started, and the delay in responding, fires/wildfires and/or public safety events are a danger to human life, personal property, the environment, localized economies, wildlife, etc. Many human lives and/or injuries can be at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

The data transmitted may be in the form of an alert or a critical alert. Further, the alerts may be communicated to utility personnel, authorized parties, first responders, operations systems, artificial intelligence platforms, etc.

Note that the general form factor fire mitigation and/or public safety event detection device 3700 may backhaul data, including data indicative of smoke and/or noxious gas sensors, infrared camera images, ambient and/or surface temperatures, humidity, nuclear radiation, seismic activity, and weather, and/or voltage drops or spikes indicative of downed, broken and/or a faulty conductor(s), etc.

Regardless of where the data originates, the general form factor fire mitigation and/or public safety event detection device 3700 backhauls the data. The general form factor fire mitigation and/or public safety event detection device 3700 transmits the data to a central computing system and/or to personnel who need the information, including utility personnel, authorized parties, first responders, artificial intelligence platforms, and/or to operations systems.

In addition, the general form factor fire mitigation and/or public safety event detection device 3700 may comprise an antenna (not shown). The antenna shall allow improved communications to the central computing device(s), operations systems, artificial intelligence platforms, authorized third parties, utilities, first responders, etc.

In one embodiment, the general form factor fire mitigation and/or public safety event detection device 3700 may comprise a magnet or bracket (not shown) on the back of the fire mitigation and/or public safety event detection device 3100. The magnet or bracket allow the general form factor fire mitigation and/or public safety event detection device 3700 to be attached to the host transformer, power pole, tree, building, host asset(s), etc.

Furthermore, the general form factor fire mitigation and/or public safety event detection device 3700 is configured to be tethered to and/or wirelessly in communication with sensors that communicate with the general form factor fire mitigation and/or public safety event detection device 3700.

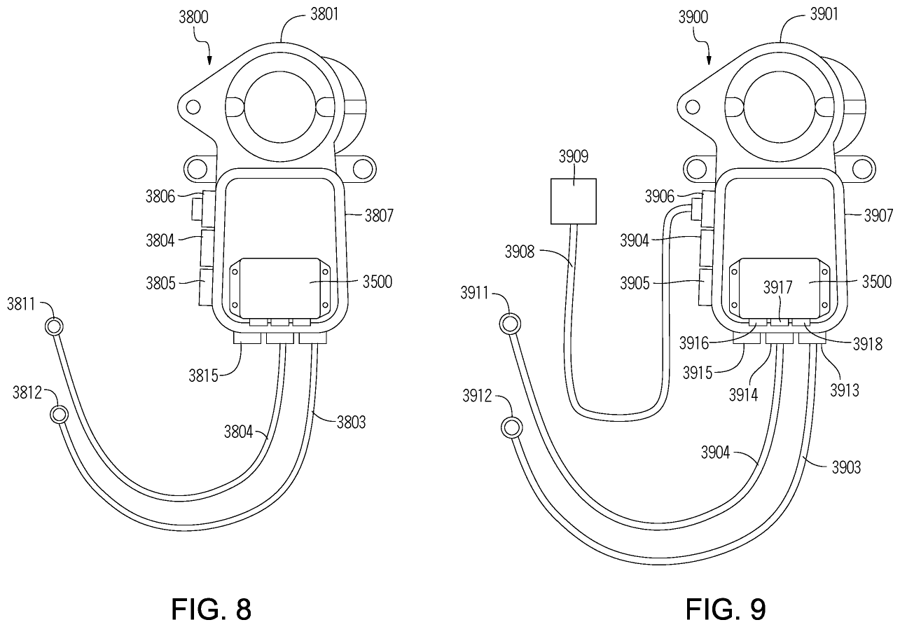

FIG. 8 is a general form factor fire mitigation and/or public safety event detection device 3800 in accordance with an embodiment of the present disclosure. The general form factor fire mitigation and/or public safety event detection device 3800 bearing onboard sensors is like the general form factor fire mitigation and/or public safety event detection device 3700 (FIG. 7). In this regard, the general form factor fire mitigation and/or public safety event detection device 3800 comprises all the functionality of the fire mitigation and/or public safety event detection device 3700.

Within a box-shaped unit 3807 resides one or more printed circuit boards (PCB) (not shown), semiconductor chips (not shown), one or more onboard sensors (not shown), and/or other electronics and/or sensors (not shown) for performing operations related to the general form factor fire mitigation and/or public safety event detection device 3800. In one embodiment, the box-shaped unit 3807 is a substantially rectangular housing; however, differently sized, and differently shaped housings may be used in other embodiments.

Different than the general form factor fire mitigation and/or public safety event detection device 3700, the general form factor fire mitigation and/or public safety event detection device 3800 comprises a coupled external sensor pack 3500. In this regard, the coupled external sensor pack 3500 comprises a plurality of sensors, and data detected by the sensors contained in the sensor pack are communicated to the electronics within the box-shaped unit 3807. The externally coupled sensor pack 3500 may also contain a plurality of sensors including, but not limited to, an infrared imaging camera, vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, and/or fault sensors, geo-positioning sensors, etc. The externally coupled sensor pack may contain one (1) or more power cables (not shown) to operate the sensor pack 3500, and/or the externally coupled sensor pack 3500 can be powered via battery which may or may not include solar, and/or via power harvesting from the host device 3801.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

Also, the general form factor fire mitigation and/or public safety event detection device 3800 comprises the voltage leads and power source conductors 3803 and 3804. These conductors are attached to a power source, e.g., a host transformer, a host asset(s), etc. The power source operates the general form factor fire mitigation and/or public safety event detection device 3800, and associated sensors.

Within the extended box-shaped unit 3807 can be a plurality of onboard sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

The general form factor fire mitigation and/or public safety event detection device 3800 further comprises ports 3805, 3806, 3804, and 3815. Other sensors may be tethered to these ports and/or wirelessly connected to these ports. The sensors may collect data regarding the localized surrounding conditions, the host transformer, the host asset(s), etc. and the surrounding environment of the general form factor fire mitigation and/or public safety event detection device 3800. Also, the general form factor fire mitigation and/or public safety event detection device 3800 comprises a port 3815 to which a sensor or a power source may be connected.

In operation, the general form factor fire mitigation and/or public safety event detection device 3800 receives power from a power source (not shown) to which the general form factor fire mitigation and/or public safety event detection device 3800 is coupled. The power operates the various sensors described above.

Further, the sensors continuously gather information about the localized surroundings, and/or the host transformer, the host asset(s), and/or the environment to determine if a public safety event and/or fire(s)/wildfire(s) conditions and/or activities, and/or useful event prevention data are detected, and/or to facilitate ongoing conditions monitoring, and or facilitate artificial intelligence platforms, and/or operations systems.

In this regard, the infrared imaging camera(s) can obtain images that indicate a public safety event and/or a fire/wildfire, and/or associated conditions. The smoke sensor can sense the presence of smoke, noxious gases, etc. If the general form factor fire mitigation and/or public safety event detection device 3800 detects voltage spikes and/or drops indicative of downed, broken and/or faulty conductors, and/or fire/wildfire and/or public safety event, the fire mitigation and/or public safety event detection device 3800 may transmit data to a monitoring device, which can be a computer, an operations system(s), artificial intelligence platform(s), etc., indicating that there is a public safety event and/or fire/wildfire present and/or conditions associated with such events are present.

The data indicating a fire/wildfire and/or public safety event is present may be transmitted in any number of communication methods. In this regard, the communication to the computer or related system(s) or platform(s) may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to utility personnel, authorized third parties, first responders, operations systems and/or artificial intelligence platforms, etc.

Regardless of how the fire/wildfire and/or public safety event started, and the delay in responding, fires/wildfires and public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or a public safety event occurs.

In addition to wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the general form factor fire mitigation and/or public safety event detection device 3800 may backhaul data, including data indicative of smoke and/or noxious gas sensors, infrared camera images, ambient and/or surface temperatures, humidity, nuclear radiation, seismic activity, voltage spikes or drops indicative of downed, broken and/or faulty conductors, weather, etc.

Regardless of where the data originates, the general form factor fire mitigation and/or public safety event detection device 3800 backhauls the data. The general form factor fire mitigation and/or public safety event detection device 3800 transmits the data to central computing systems, personnel, including utility personnel, authorized third parties, and/or first responders, and/or to operations systems and/or artificial intelligence platforms, etc.

In addition, the general form factor fire mitigation and/or public safety event detection device 3800 may comprise an antenna (not shown). The antenna shall allow improved communications to the central computing device(s), operations systems, artificial intelligence platforms, authorized third parties, utilities, first responders, etc.

In one embodiment, the general form factor fire mitigation and/or public safety event detection device 3800 may comprise a magnet or bracket (not shown) on the back of the general form factor fire mitigation and/or public safety event detection device 3800. The magnet or bracket allow the general form factor fire mitigation and/or public safety event detection device 3800 to be attached to the host transformer, host asset(s), a tree, building, etc.

Furthermore, the general form factor fire mitigation and/or public safety event detection device 3800 is configured to be tethered to and/or wirelessly in communication with sensors that communicate with the fire mitigation and/or public safety event detection device 3800.

FIG. 9 is a general form factor fire mitigation and/or public safety event detection device 3900 in accordance with an embodiment of the present disclosure. The general form factor fire mitigation and/or public safety event detection device 3900 is like the general form factor fire mitigation and/or public safety event detection device 3800 (FIG. 8). In this regard, the general form factor fire mitigation and/or public safety event detection device 3900 comprises all the functionality of the fire mitigation and/or public safety event detection device 3800. However, in addition, the general form factor fire mitigation and/or public safety event detection device 3900 also comprises an external wired sensor pack 3909 tethered to the general form factor fire mitigation and/or public safety event detection device 3900 via a cable 3908, which can measure other data like external temperatures, etc. Further, the general form factor mitigation device 3900 comprises and externally coupled sensor pack 3917.

The general form factor fire mitigation and/or public safety event detection device 3900 comprises an extended box-shaped unit 3907 bearing onboard sensors. Within the extended box-shaped unit 3907 resides one or more printed circuit boards (PCB) (not shown), semiconductor chips (not shown), and/or other electronics and/or sensors (not shown) for performing operations related to the extended box-shaped unit 3907. In one embodiment, the extended box-shaped unit 3907 is a substantially rectangular housing; however, differently sized, and differently shaped housings may be used in other embodiments.

An externally coupled sensor pack 3500 may also contain a plurality of sensors including, but not limited to, an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors, etc. The externally coupled sensor pack may contain one (1) or more power cables to operate the sensor pack 3500, and/or the externally coupled sensor pack 3500 can be powered via battery which may or may not include solar, and/or via power harvesting from the host device 3901.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

The externally coupled sensor pack 3500 comprises ports 3916-3918. The ports 3916-3918 may couple to other sensors (not shown), power supplies (not shown), and/or other devices.

Additionally, the general form factor fire mitigation and/or public safety event detection device 3900 further comprises one or more cables 3904, 3903. The power source and/or voltage cables 3904, 3903 on extended box-shaped unit 3907 may be coupled to a an power source such as a conductor cable or corresponding bus bars or bushings (not shown) and ground or reference voltage conductor(s) (not shown), respectively, for the corresponding power source and/or voltage cable with terminators 3911, 3912, respectively. The cables 3904, 3903 provide power to the extended box-shaped unit 3907, and/or detect voltage data.

Within the extended box-shaped unit 3907 are a plurality of onboard sensors. The sensors may include, but are not limited to, an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

The general form factor fire mitigation and/or public safety event detection device 3900 differs from fire mitigation and/or public safety event detection device 3800. In this regard, the general form factor fire mitigation and/or public safety event detection device 3900 comprises an external wired sensor pack 3909 to which an external wired sensor pack 3909 may be coupled via cable 3908. The sensor pack 3909 may gather, for example, the external temperature of the host transformer, and/or other sensor data points.

In operation, the general form factor fire mitigation and/or public safety event detection device 3900 receives power from a host transformer (not shown), or host asset(s) (not shown) from voltage leads also serving as power cables 3903 and 3904 to which the general form factor fire mitigation and/or public safety event detection device 3900 is coupled. The power operates the various sensors described above.

Further, the sensors continuously gather information about the localized surrounding area, the host transformer, the host asset(s), and the environmental surroundings to determine if a public safety event and/or fire(s)/wildfire(s) conditions and/or activities exist, and/or useful event prevention data are detected, and/or to facilitate ongoing conditions monitoring, and/or to support operations systems and/or artificial intelligence platforms, etc.

In this regard, the infrared imaging camera(s) can obtain images that indicate a fire/wildfire and/or public safety event, or associated conditions. The smoke sensor can sense the presence of smoke, noxious gases, etc. If the general form factor fire mitigation and/or public safety event detection device 3900 detects fire/wildfire and/or a public safety event including but not limited to a broken, downed or faulty conductor, nuclear radiation, seismic activity, etc., the fire mitigation and/or public safety event detection device 3900 may transmit data to a monitoring device, which can be a computer, operations system(s), artificial intelligence platform(s), etc. indicating that there is a public safety event and/or fire/wildfire present, and/or associated conditions are present.

The data indicating a public safety and/or fire/wildfire is present and/or associated conditions are present may be transmitted in any number of communication methods. In this regard, the communication to the computer may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to a central computing device(s), operations system(s), artificial intelligence platform(s), utility personnel, authorized parties, and/or first responders.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the general form factor fire mitigation and/or public safety event detection device 3900 may backhaul data, including data indicative of smoke and/or noxious gas sensors, infrared camera images, ambient and/or surface temperatures, humidity, nuclear radiation, seismic activity, and weather, and/or voltage drops or spikes indicative of downed, broken and/or a faulty conductor(s), etc.

Regardless of where the data originates, the fire mitigation and/or public safety event detection device 3900 backhauls the data. The general form factor fire mitigation and/or public safety event detection device 3900 transmits the data to a central computing system, and/or to authorized personnel including utility personnel, authorized parties, and/or first responders, and/or to operations systems and/or to artificial intelligence platforms, etc.

In addition, the general form factor fire mitigation and/or public safety event detection device 3900 may comprise an antenna (not shown). The antenna shall allow improved communications to the central computing device(s), operations systems, artificial intelligence platforms, authorized third parties, utilities, first responders, etc.

In one embodiment, the general form factor fire mitigation and/or public safety event detection device 3900 may comprise a magnet or bracket (not shown) on the back of the general form factor fire mitigation and/or public safety event detection device 3900. The magnet or bracket allow the general form factor fire mitigation and/or public safety event detection device 3900 to be attached to a host transformer, host asset(s), a tree, building, power pole, etc.

Furthermore, the general form factor fire mitigation and/or public safety event detection device 3900 is configured to be tethered to and/or wirelessly in communication with sensors that communicate with the fire mitigation and/or public safety event detection device 3900.

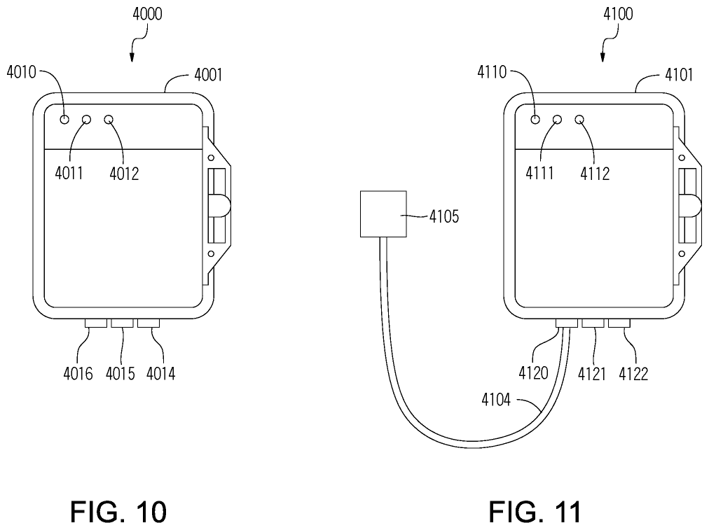

FIG. 10 is a fire mitigation and/or public safety event detection device 4000 bearing onboard sensors in accordance with an embodiment of the present disclosure. The fire mitigation and/or public safety event detection device 4000 comprises a housing 4001. The fire mitigation and/or public safety event detection device 4000 does not have voltage leads or power cables. Instead, the fire mitigation and/or public safety event detection device 4000 is powered by a battery (not shown) within housing 4001 which may or may not include solar capability.

Within the housing 4001 are a plurality of onboard sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

The fire mitigation and/or public safety event detection device 4000 further comprises ports 4014-4016. Additional sensors may be tethered to and/or wirelessly connected to the fire mitigation and/or public safety event detection device 4000 through these ports 4014-4016.

In operation, the fire mitigation and/or public safety event detection device 4000 receives power from a battery (not shown). The battery power operates the various sensors described above.

Further, the sensors continuously gather information about the localized surrounding area, and environmental conditions, and/or the host deployment asset(s) to determine if a public safety event and/or fire(s)/wildfire(s) conditions and/or activities, and/or useful event prevention data are detected, and/or to facilitate ongoing host asset and surrounding conditions monitoring.

In this regard, the infrared imaging camera(s) can obtain images that indicate a fire/wildfire and/or a public safety event, and/or associated conditions. The smoke sensor can sense the presence of smoke, noxious gases, etc. If the fire mitigation and/or public safety event detection device 4000 detects a public safety event, and/or a fire/wildfire, the fire mitigation and/or public safety event detection device 4000 transmits data to a monitoring device, which can be a computer, operations system(s), artificial intelligence platform(s), etc. indicating that there is a public safety event and/or fire/wildfire present, and/or associated conditions are detected.

The data indicating a fire/wildfire and/or public safety event is present may be transmitted in any number of communication methods. In this regard, the communication to the computer, operations system(s), artificial intelligence platform(s), and/or authorized parties may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to a central computing system, operations systems, artificial intelligence platforms, etc. and/or to utility personnel, approved third parties, and/or first responders, etc.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the fire mitigation and/or public safety event detection device 4000 may backhaul data, including data indicative of smoke and/or noxious gas sensors, infrared camera images, ambient and/or surface temperatures, humidity, nuclear radiation, seismic activity, weather, etc.

Regardless of where the data originates, the fire mitigation and/or public safety event detection device 4000 backhauls the data. The fire mitigation and/or public safety event detection device 4000 transmits the data to a central computing system(s) and/or to personnel, including utility personnel, authorized third parties, and/or first responders, and/or to operations systems, and/or to artificial intelligence platforms, etc.

In addition, the fire mitigation and/or public safety event detection device 4000 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4000 may comprise a magnet or bracket (not shown) on the back of the fire mitigation and/or public safety event detection device 4000. The magnet or bracket allow the fire mitigation and/or public safety event detection device 4000 to be attached to a power pole, a transmission tower, a host transformer, a tree, a building, etc.

FIG. 11 is a fire mitigation and/or public safety event detection device 4100 bearing onboard sensors in accordance with an embodiment of the present disclosure. The fire mitigation and/or public safety event detection device 4100 is like the fire mitigation and/or public safety event detection device 4000 (FIG. 10) in that it is powered by a battery (not shown) within housing 4101 which may or may not include solar capability. In this regard, the fire mitigation and/or public safety event detection device 4100 comprises a housing 4101.

The difference between fire mitigation and/or public safety event detection device 4000 (FIG. 10) and fire mitigation and/or public safety event detection device 4100 is that the fire mitigation and/or public safety event detection device 4100 comprises an external wired sensor pack 4105. The external wired sensor pack 4105 can detect such characteristics as temperature, smoke, etc. Upon collection of data by the external sensor 4105, the external sensor 4105 transmits the data to the housing 4101 via a cable 4104.

Within the housing 4101 are a plurality of onboard sensors. The sensors may include, but are not limited to, an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

The fire mitigation and/or public safety event detection device 4100 may comprise a plurality of ports 4120-4122, and 4112. Communication mediums or external sensors may be connected to the ports 4120-4122, and 4112, wired or wirelessly.

Further, the sensors continuously gather information about the localized surrounding area, and/or the host transformer, and/or the host asset(s), and the environmental surroundings to determine if a public safety event and/or fire(s)/wildfire(s) conditions and/or activities, and/or useful event prevention data are detected, and/or to facilitate ongoing surroundings and conditions monitoring.

In this regard, the infrared imaging camera(s) can obtain images that indicate a fire/wildfire and/or public safety event, or associated conditions. The smoke sensor can sense the presence of smoke, noxious gases, etc. Other sensors may detect humidity changes, ambient or other temperature changes, the presence of nuclear radiation, seismic activity, etc. If the fire mitigation and/or public safety event detection device 4100 detects a public safety event and/or fire/wildfire, the fire mitigation and/or public safety event detection device 4100 may transmit data and/or alert messages to a monitoring device, which can be a computer, operations system(s), artificial intelligence platform(s), authorized third parties, etc. indicating that there is a fire/wildfire and/or public safety event present, and/or associated conditions are present.

The data indicating a public safety event and/or a fire/wildfire is present and/or associated conditions are present may be transmitted in any number of communication methods. In this regard, the communication to the computer and/or the systems, platforms and/or personnel may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to a central computing device(s), operations system(s), artificial intelligence platform(s), utility personnel, authorized third parties, and/or first responders, etc.

Note that the fire mitigation and/or public safety event detection device 4100 may backhaul data, including data indicative of smoke and/or noxious gas sensors, infrared camera images, ambient and/or surface temperatures, humidity, nuclear radiation, seismic activity, and/or weather, etc.

Regardless of where the data originates, the fire mitigation and/or public safety event detection device 4100 backhauls the data. The fire mitigation and/or public safety event detection device 4100 transmits the data to a central computing device, authorized personnel including utility personnel, authorized third parties, and/or first responders, and/or to operations systems, and/or artificial intelligence platforms, etc.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

In addition, the fire mitigation and/or public safety event detection device 4100 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4100 may comprise a magnet or bracket (not shown) on the back of the fire mitigation and/or public safety event detection device 4100. The magnet or bracket allow the fire mitigation and/or public safety event detection device 4100 to be attached to a power pole, a tree, a building, the host transformer, the host asset(s), etc.

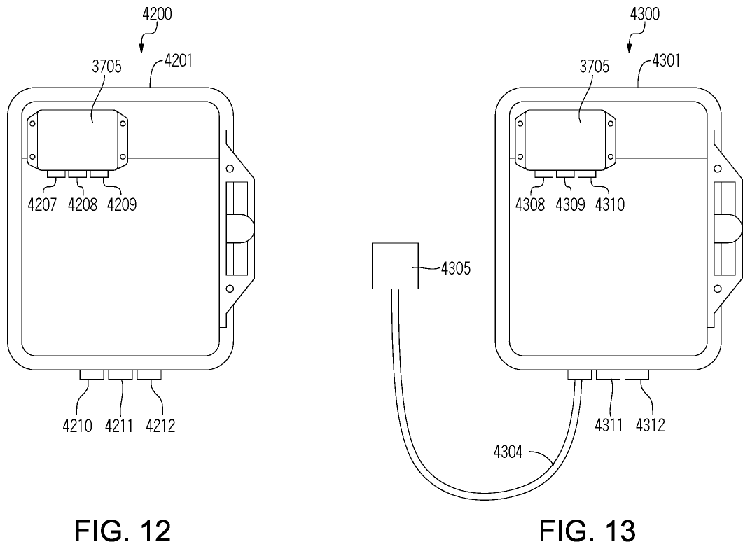

FIG. 12 is a fire mitigation and/or public safety event detection device 4200 bearing onboard sensors in accordance with an embodiment of the present disclosure. The fire mitigation and/or public safety event detection device 4200 is like the fire mitigation and/or public safety event detection device 4000 (FIG. 10), in that it is powered by a battery (not shown) within housing 4201 which may or may not include solar capability. In this regard, the fire mitigation and/or public safety event detection device 4200 comprises a housing 4201.

The difference between fire mitigation and/or public safety event detection device 4200 and fire mitigation and/or public safety event detection device 4000 is that the fire mitigation and/or public safety event detection device 4200 comprises an externally coupled sensor pack 3705.

Within the housing 4201 are a plurality of sensors. The onboard sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

Additionally, the fire mitigation and/or public safety event detection device 4200 comprises the externally coupled sensor pack 3705. In this regard, the externally coupled sensor pack 3705 may comprise a plurality of sensors, and data detected by the sensor(s) contained in the externally coupled sensor pack are communicated to the electronics within the housing 4201. The externally coupled sensor pack may be powered by battery which may or may not include solar, and/or via the use of 1 or more power cables (not shown).

The sensor pack 3705 may comprise a plurality of ports 4207, 4208, and 4209.

Communication mediums or external sensors may be connected to the ports 4207, 4208, and 4209. In this embodiment, the sensor pack 3705 does not comprise an external power source. Instead, the external sensor pack 3705 comprises a battery (not shown) that may be solar powered.

Also, the fire mitigation and/or public safety event detection device 4200 comprises a plurality of ports 4210, 4211, and 4212. The ports 4210, 4211, and 4212 may be coupled to additional sensors (not shown) or to a power source (not shown).

Further, the sensors continuously gather information about the localized surrounding conditions, the host transformer, the host asset(s), and the environmental surroundings to determine if a public safety event and/or fire(s)/wildfire(s) conditions and/or activities, and/or useful event prevention data are detected, and/or to facilitate ongoing environmental and/or localized conditions monitoring.

In this regard, the infrared imaging camera(s) can obtain images that indicate a fire/wildfire and/or public safety event. The smoke sensor can sense the presence of smoke, noxious gases, etc. Other sensors may detect humidity changes, ambient or other temperature changes, the presence of nuclear radiation, seismic activity, etc. If the fire mitigation and/or public safety event detection device 4200 detects a public safety event and/or fire/wildfire, and/or associated conditions, the fire mitigation and/or public safety event detection device 4200 may transmit data and/or alert messages to a monitoring device, which can be a computer, operations system(s), artificial intelligence platform(s), authorized third parties, etc. indicating that there is a fire/wildfire and/or public safety event present, and/or associated conditions are present.

The data indicating a public safety event and/or a fire/wildfire is present may be transmitted in any number of communication methods. In this regard, the communication to the computer may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to a central computing system, operations systems, artificial intelligence platforms, utility personnel, authorized third parties, and/or first responders, etc.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or a public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the fire mitigation and/or public safety event detection device 4200 may backhaul data, including data indicative of smoke and/or noxious gas sensors, infrared camera images, ambient and/or surface temperatures, humidity, nuclear radiation, seismic activity, and/or weather, etc.

Regardless of where the data originates, the fire mitigation and/or public safety event detection device 4200 backhauls the data. The fire mitigation and/or public safety event detection device 4200 transmits the data to central computing systems, operations systems, artificial intelligence platforms, personnel including utility personnel, authorized third parties, and/or first responders, etc.

In addition, the fire mitigation and/or public safety event detection device 4200 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4200 may comprise a magnet or bracket (not shown) on the back of the fire mitigation and/or public safety event detection device 4200. The magnet or bracket allow the fire mitigation and/or public safety event detection device 4200 to be attached to a power pole, a tree, a building the host transformer, the host asset(s), and/or another outdoor object, etc.

FIG. 13 is a fire mitigation and/or public safety event detection device 4300 bearing onboard sensors in accordance with an embodiment of the present disclosure. The fire mitigation and/or public safety event detection device 4300 is like the fire mitigation and/or public safety event detection device 4100 (FIG. 11), in that it is powered by a battery (not shown) within housing 4301 which may or may not include solar capability. In this regard, the fire mitigation and/or public safety event detection device 4300 comprises a housing 4301.

The difference between fire mitigation and/or public safety event detection device 4300 and fire mitigation and/or public safety event detection device 4200 is that the fire mitigation and/or public safety event detection device 4200 comprises an external wired sensor pack 4305. The external wired sensor pack 4305 collects data, e.g., temperature, smoke, etc. The external sensor pack 4305 transmits data indicative of what is detected to the housing 4301.

Within the housing 4301 are a plurality of sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

Additionally, the fire mitigation and/or public safety event detection device 4300 comprises the externally coupled sensor pack 3705. In this regard, the externally coupled sensor pack 3705 may comprise a plurality of sensors, and data detected by the sensor(s) contained in the external coupled sensor pack are communicated to the electronics within the housing 4301. The externally coupled sensor pack may be battery powered, which may or may not include solar power, and/or one (1) or more power cables can be used to attach the externally coupled sensor pack 3705 to a host power source.

The sensor pack 3705 may comprise a plurality of ports 4308, 4309, and 4310. Communication mediums or external sensors may be connected to the ports 4308, 4309, and 4310. In this embodiment, the sensor pack 3705 does not comprise an external power source. Instead, the external sensor pack 3705 comprises a battery (not shown) that may be solar powered.

In addition, the fire mitigation and/or public safety event detection device 4300 also does not have an external power source. Instead, there is a battery (not shown) contained in the housing 4301 for powering the electronics and sensors in the housing 4301. The battery may also be configured to power sensors in the external sensor pack 3705.

Further, the sensors continuously gather information about the localized surrounding conditions, the host transformer, the host asset(s), and the surroundings environment to determine if a public safety event and/or fire(s)/wildfire(s) conditions and/or activities, and/or useful event prevention data are detected, and/or to facilitate ongoing surroundings and environmental conditions monitoring.

In this regard, the infrared imaging camera(s) can obtain images that indicate a fire/wildfire and/or public safety event, and/or associated conditions. The smoke sensor can sense the presence of smoke, noxious gases, etc. Other sensors may detect humidity changes, ambient or other temperature changes, the presence of nuclear radiation, seismic activity, etc. If the fire mitigation and/or public safety event detection device 4300 detects a public safety event and/or fire/wildfire, the fire mitigation and/or public safety event detection device may transmit data and/or alert messages to a monitoring device, which can be a computer, operations systems, artificial intelligence platforms, authorized third parties, etc. indicating that there is a fire/wildfire and/or public safety event is present, and/or associated conditions are present.

The data indicating a public safety event and/or a fire/wildfire is present, or associated conditions are present, may be transmitted in any number of communication methods. In this regard, the communication to the computer, operating systems, artificial intelligence platforms, authorized third parties, etc. may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to a central computing system, operations systems, artificial intelligence platforms, utility personnel, authorized third parties, and/or first responders, etc.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the fire mitigation and/or public safety event detection device 4300 may backhaul data, including data indicative of smoke and/or noxious gas sensors, infrared camera images, ambient and/or surface temperatures, humidity, nuclear radiation, seismic activity, and/or weather, etc.

Regardless of where the data originates, the fire mitigation and/or public safety event detection device 4300 backhauls the data. The fire mitigation and/or public safety event detection device 4300 transmits the data to central computing devices, operations systems, artificial intelligence platforms, personnel including utility personnel, authorized third parties, and/or first responders, etc.

In addition, the fire mitigation and/or public safety event detection device 4300 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4300 may comprise a magnet or bracket (not shown) on the back of the fire mitigation and/or public safety event detection device 4300. The magnet or bracket allow the fire mitigation and/or public safety event detection device 4300 to be attached to a power pole, a building, a host asset(s), the host transformer, and/or another outdoor object, e.g., a tree, etc.

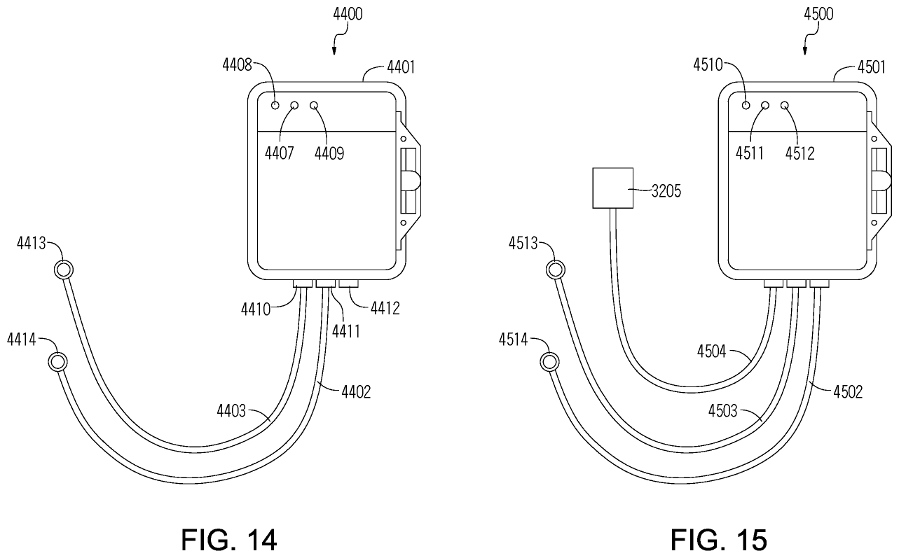

FIG. 14 is a fire mitigation and/or public safety event detection device 4400 bearing onboard sensors in accordance with an embodiment of the present disclosure. The fire mitigation and/or public safety event detection device 4400 is like the fire mitigation and/or public safety event detection device 4000 (FIG. 10). In this regard, the fire mitigation and/or public safety event detection device 4400 comprises a housing 4401.

The fire mitigation and/or public safety event detection device 4400 comprises one or more cables 4402, 4403. The cables 4402, 4403 may be coupled to a conductor cable or corresponding bus bars (not shown) and ground or reference voltage conductor (not shown), respectively, for the corresponding conductor cable with terminators 4414, 4413, respectively. The cables 4402, 4403 provide power to the fire mitigation and/or public safety event detection device 4400, and/or may be used to attain voltage information.

Within the housing 4401 is a plurality of onboard sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

In operation, the fire mitigation and/or public safety event detection device 4400 receives power from a host transformer or host asset (not shown) to which the fire mitigation and/or public safety event detection device 4400 is coupled by cables 4402 and 4403. The power operates the various sensors described above.

The fire mitigation and/or public safety event detection device 4400 also comprises two light-emitting diodes (LED) 4407 and 4408, and an additional port 4412. The LEDs 4407 and 4408 can communicate the status of the fire mitigation and/or public safety event detection device 4400. For example, if the lights are green, that may indicate that the general form factor fire mitigation and/or public safety event detection device 4400 is operating properly. However, if the lights are red, that may indicate that the general form factor fire mitigation and/or public safety event detection device 4400 is not operating.

Further, the fire mitigation and/or public safety event detection device 4400 comprises an optical port 4409, and an additional port 4412. The optical port 4409 and additional port 4412 connects with other sensors (not shown) and other devices (not shown).

Further, the sensors continuously gather information about the localized surroundings, the host transformer, the host asset(s), and the environmental surroundings to determine if a public safety event and/or fire(s)/wildfire(s) conditions and/or activities, and/or useful event prevention data are detected, and/or to facilitate ongoing asset and environmental conditions monitoring.

In this regard, the infrared imaging camera(s) can obtain images that indicate a fire/wildfire and/or a public safety event. The smoke sensor can sense the presence of smoke, and/or noxious gases, etc. The voltage sensor capability enables the detection of voltage drops or spikes indicative of broken, downed and/or faulty conductors. Other sensors may detect the presence of nuclear radiation, seismic activity, humidity change, etc. If the fire mitigation and/or public safety event detection device 4400 detects fire, smoke, voltage issues, etc. as indicated above, the fire mitigation and/or public safety event detection device 4400 may transmit data to a monitoring device, which can be a computer, operations system(s), artificial intelligence platform, cell phone, authorized third parties, etc. indicating that there is a public safety event and/or fire/wildfire present, and/or associated conditions are present.

The data indicating a public safety and/or fire/wildfire is present may be transmitted in any number of communication methods. In this regard, the communication to the computer, operating system(s), artificial intelligence platform(s), authorized third parties, etc. may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to utility personnel, authorized third parties, and/or first responders.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the fire mitigation and/or public safety event detection device 4400 may backhaul data, including data indicative of smoke and/or noxious gas sensors, infrared camera images, ambient and/or surface temperatures, humidity, nuclear radiation, seismic activity, voltage issues, transformer can temperatures, and weather, etc.

Regardless of where the data originates, the fire mitigation and/or public safety event detection device 4400 backhauls the data. The fire mitigation and/or public safety event detection device 4400 transmits the data to a central computing system(s), and/or operations systems, and/or artificial intelligence platforms, and/or authorized personnel, including utility personnel first responders, etc.

In addition, fire mitigation and/or public safety event detection device 4400 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4400 may comprise a magnet or bracket (not shown) on the back of the fire mitigation and/or public safety event detection device 4400. The magnet or bracket allow the fire mitigation and/or public safety event detection device 4400 to be attached to a power pole, a transmission tower, a building, the host transformer, the host asset(s), a tree, etc.

FIG. 15 is a fire mitigation and/or public safety event detection device 4500 bearing onboard sensors in accordance with an embodiment of the present disclosure. The fire mitigation and/or public safety event detection device 4500 is like the fire mitigation and/or public safety event detection device 4400 (FIG. 14). In this regard, the fire mitigation and/or public safety event detection device 4500 comprises a housing 4501.

The fire mitigation and/or public safety event detection device 4500 comprises one or more power and/or voltage cables 4502, 4503. The cables 4502, 4503 may be coupled to a conductor cable or corresponding bus bars (not shown) and ground or reference voltage conductor (not shown), respectively, for the corresponding conductor cable with terminators 4514, 4513, respectively. The cables 4502, 4503 provide power to the fire mitigation and/or public safety event detection device 4500, and/or may be used to attain voltage information.

Within the housing 4501 is a plurality of sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

Additionally, the fire mitigation and/or public safety event detection device 4500 is coupled to an external wired sensor pack 3205 via a cable 4504. The external sensor pack 3205 can detect, for example, transformer can temperature, and/or other sensor data points. Upon detection, the external sensor pack 4505 can transmit data indicative of the sensor data point to the housing 4501.

In operation, the fire mitigation and/or public safety event detection device 4500 receives power from a host transformer or host asset(s) (not shown) to which the fire mitigation and/or public safety event detection device 4500 is coupled by cables 4502 and 4503. The power operates the various sensors described above. The cables 4502 and 4503 may also serve as voltage leads.

The fire mitigation and/or public safety event detection device 4500 also comprises two light-emitting diodes (LED) 4510 and 4511. The LEDs 4510 and 4511 can communicate the status of the fire mitigation and/or public safety event detection device 4500. For example, if the lights are green, that may indicate that the general form factor fire mitigation and/or public safety event detection device 4500 is operating properly. However, if the lights are red, that may indicate that the general form factor fire mitigation and/or public safety event detection device 4500 is not operating.

Further, the fire mitigation and/or public safety event detection device 4500 comprises an optical port 4512. The optical port 4512 connects with other sensors (not shown) and other devices (not shown).

Further, the sensors continuously gather information about the localized surroundings, the host transformer, host asset(s), and the environmental surroundings to determine if a public safety event and/or fire(s)/wildfire(s) conditions and/or activities, and/or useful event prevention data are detected, and/or to facilitate ongoing asset and environmental conditions monitoring.

In this regard, the infrared imaging camera(s) can obtain images that indicate a fire/wildfire and/or public safety event, and/or associated conditions. The smoke sensor can sense the presence of smoke, and/or noxious gases, etc. The voltage sensor capability enables the detection of voltage drops or spikes indicative of broken, downed and/or faulty conductors. Other sensors may detect the presence of nuclear radiation, seismic activity, humidity change, etc. If the fire mitigation and/or public safety event detection device 4500 detects fire, smoke, voltage issues, etc. as indicated above, the fire mitigation and/or public safety event detection device 4500 may transmit data to a monitoring device, which can be a computer, operations system(s), artificial intelligence platform(s), authorized third parties, cell phone, etc. indicating that there is a public safety event and/or fire/wildfire present, and/or associated conditions are present.

The data indicating a public safety and/or fire/wildfire is present may be transmitted in any number of communication methods. In this regard, the communication to the computer, operating system(s), artificial intelligence platform(s), authorized third parties, etc. may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to utility personnel, authorized third parties, and/or first responders.

Regardless of how the fire/wildfire and/or public safety event started, and the delay in responding, fires/wildfires and/or public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or a public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

Note that the fire mitigation and/or public safety event detection device 4500 may backhaul data, including data indicative of smoke and/or noxious gas sensors, infrared camera images, ambient and/or surface temperatures, humidity, nuclear radiation, seismic activity, transformer can temperatures, voltage issues, and weather, etc.

Regardless of where the data originates, the fire mitigation and/or public safety event detection device 4500 backhauls the data. The fire mitigation and/or public safety event detection device 4500 transmits the data to a central computing device, operations systems, artificial intelligence platforms, authorized personnel including utility personnel, first responders, etc.

In addition, the fire mitigation and/or public safety event detection device 4500 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4500 may comprise a magnet or bracket (not shown) on the back of the fire mitigation and/or public safety event detection device 4500. The magnet or bracket allow the fire mitigation and/or public safety event detection device 4500 to be attached to a power pole, a transmission tower, a building, the host transformer, the host asset(s), or other outdoor objects, e.g., a tree, etc.

FIG. 16 is a fire mitigation and/or public safety event detection device 4600 bearing onboard sensors in accordance with an embodiment of the present disclosure. The fire mitigation and/or public safety event detection device 4600 is like the fire mitigation and/or public safety event detection device 4400 (FIG. 14). In this regard, the fire mitigation and/or public safety event detection device 4600 comprises a housing 4601.

The difference between fire mitigation and/or public safety event detection device 4600 and fire mitigation and/or public safety event detection device 4400 is that the fire mitigation and/or public safety event detection device 4600 comprises an externally coupled sensor pack 3705.

In this regard, the externally coupled sensor pack 3705 may comprise a plurality of sensors, and data detected by the sensor(s) contained in the externally coupled sensor pack 3705 are communicated to the electronics within the housing 4301. The externally coupled sensor pack may be battery powered, which may or may not include solar power, and/or one (1) or more power cables can be used to attach the externally coupled sensor pack 3705 to a host power source.

The sensor pack 3705 may comprise a plurality of ports 4601, 4602, and 4603.

Communication mediums or external sensors may be connected to the ports 4601, 4602, and 4603. In this embodiment, the sensor pack 3705 does not comprise an external power source. Instead, the external sensor pack 3705 comprises a battery (not shown) that may be solar powered.

The fire mitigation and/or public safety event detection device 4600 comprises one or more cables 4602, 4603. The cables 4602, 4603 may be coupled to a conductor cable or corresponding bus bars (not shown) and ground or reference voltage conductor (not shown), respectively, for the corresponding conductor cable with terminators 4614, 4613, respectively. The cables 4602, 4603 provide power to the fire mitigation and/or public safety event detection device 4600, and/or serve as voltage leads from which voltage information may be attained.

Within the housing 4601 is a plurality of sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Note that the infrared imaging discloses a heat differential between the objects on fire and the objects not on fire, and/or other unusual temperature differentials occurring below and/or nearby the respective fire mitigation and/or public safety event detection device(s). In this regard, the infrared camera(s) capturing the data may have a wide field of view (and the other infrared cameras in the multi-device deployed web of fire mitigation and/or public safety event detection devices). Thus, the data from each infrared camera(s) in the web of fire mitigation and/or public safety event detection devices provides a different perspective of the fire/wildfire and/or public safety event(s). The collage or matrix of images presented by the infrared camera(s) can provide utility personnel, third parties, or first responders a more complete picture of the fire/wildfire and/or public safety event(s).

Further, the sensors continuously gather information about the localized surroundings, the host transformer, host asset(s), and the environmental surroundings to determine if a public safety event and/or fire(s)/wildfire(s) conditions and/or activities, and/or useful event prevention data are detected, and/or to facilitate ongoing asset and environmental conditions monitoring.

In this regard, the infrared imaging camera(s) can obtain images that indicate a fire/wildfire and/or a public safety event, and/or associated conditions. The smoke sensor can sense the presence of smoke, and/or noxious gases, etc. The voltage sensor capability enables the detection of voltage drops or spikes indicative of broken, downed and/or faulty conductors. Other sensors may detect the presence of nuclear radiation, seismic activity, humidity change, etc. If the fire mitigation and/or public safety event detection device 4600 detects fire, smoke, voltage issues, etc. as indicated above, the fire mitigation and/or public safety event detection device 4600 may transmit data to a monitoring device, which can be a computer, operations system(s), artificial intelligence platform(s), authorized third parties, cell phone, etc. indicating that there is a public safety event and/or fire/wildfire present, and/or associated conditions.

The data indicating a public safety event and/or fire/wildfire is present may be transmitted in any number of communication methods. In this regard, the communication to the computer operations systems, artificial intelligence platforms, authorized third parties, etc. may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to a central computing system, operations system(s), artificial intelligence platform(s), authorized personnel such as utility personnel, authorized third parties, and/or first responders, etc.

Note that the fire mitigation and/or public safety event detection device 4600 may backhaul data, including data indicative of smoke and/or noxious gas sensors, infrared camera images, ambient and/or surface temperatures, humidity, nuclear radiation, seismic activity, transformer can temperatures, voltage issues, and weather, etc.

Regardless of where the data originates, the fire mitigation and/or public safety event detection device 4600 backhauls the data. The fire mitigation and/or public safety event detection device 4600 transmits the data to a central computing system, operations system(s), artificial intelligence platform(s), authorized personnel including but not limited to utility personnel, first responders, etc.

In addition, the fire mitigation and/or public safety event detection device 4600 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4600 may comprise a magnet or bracket (not shown) on the back of the fire mitigation and/or public safety event detection device 4600. The magnet or bracket allow the fire mitigation and/or public safety event detection device 4600 to be attached to a power pole, a transmission tower, a building, the host transformer, the host asset(s), or other outdoor objects, e.g., a tree, etc.

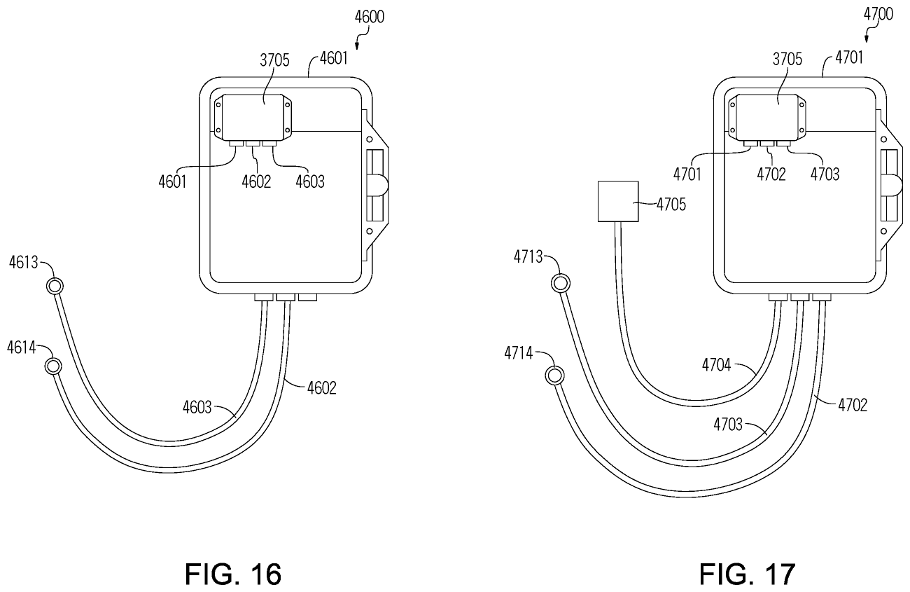

FIG. 17 is a fire mitigation and/or public safety event detection device 4700 bearing onboard sensors in accordance with an embodiment of the present disclosure. The fire mitigation and/or public safety event detection device 4700 is like the fire mitigation and/or public safety event detection device 4600 (FIG. 16). In this regard, the fire mitigation and/or public safety event detection device 4700 comprises a housing 4701.

The fire mitigation and/or public safety event detection device 4700 comprises one or more cables 4702, 4703. The cables 4702, 4703 may be coupled to a conductor cable or corresponding bus bars (not shown) and ground or reference voltage conductor (not shown), respectively, for the corresponding conductor cable with terminators 3213, 3214, respectively. The cables 4702, 4702 provide power to the fire mitigation and/or public safety event detection device 4700, and/or may be used to attain voltage information

Additionally, the fire mitigation and/or public safety event detection device 4700 comprises the externally coupled sensor pack 3705. In this regard, the externally coupled sensor pack 3705 may comprise a plurality of sensors, and data detected by the sensor(s) contained in the externally coupled sensor pack are communicated to the electronics within the housing 4701. The externally coupled sensor pack 3705 may be battery powered, which may or may not include solar power, and/or one (1) or more power cables can be used to attach the externally coupled sensor pack 3705 to a host power source.

The sensor pack 3705 may comprise a plurality of ports 4701, 4702, and 4703.

Communication mediums or external sensors may be connected to the ports 4701, 4702, and 4703. In this embodiment, the sensor pack 3705 does not comprise an external power source. Instead, the external sensor pack 3705 comprises a battery (not shown) that may be solar powered or it may pull power from a battery (not shown) located in the housing 4701.

Within the housing 4701 is a plurality of sensors. The sensors may include, but are not limited to, an infrared imaging camera(s), vibration sensors, ambient temperature sensors, seismic activity sensors, air quality sensors, environmental wind direction and speed sensors, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

Additionally, the fire mitigation and/or public safety event detection device 4700 is coupled to an external wired sensor pack 4705 via a cable 4704. The external wired sensor pack 4705 can detect, for example, transformer can temperature, and/or other sensor data points. Upon detection, data indicative of what is detected by the external wired sensor pack 4705 is transmitted to the housing 4701.

Further, the sensors continuously gather information about the localized surroundings, the host transformer, the host asset(s), and the environmental surroundings to determine if a public safety event and/or fire(s)/wildfire(s) conditions and/or activities, and/or useful event prevention data are detected, and/or to facilitate ongoing asset and environmental conditions monitoring.

In this regard, the infrared imaging camera(s) can obtain images that indicate a fire/wildfire and/or public safety event, and/or associated conditions. The smoke sensor can sense the presence of smoke, and/or noxious gases, etc. The voltage sensor capability enables the detection of voltage drops or spikes indicative of broken, downed and/or faulty conductors. Other sensors may detect the presence of nuclear radiation, seismic activity, humidity change, external transformer temperature, etc. If the fire mitigation and/or public safety event detection device 4700 detects fire, smoke, voltage issues, etc. as indicated above, the fire mitigation and/or public safety event detection device 4700 may transmit data to a monitoring device, which can be a computer, operations system(s), artificial intelligence platform(s), authorized third parties, cell phone, etc. indicating that there is a public safety event and/or fire/wildfire present, and/or associated conditions.

The data indicating a public safety and/or fire/wildfire, and/or associated conditions is present may be transmitted in any number of communication methods. In this regard, the communication to the computer, operating system(s), artificial intelligence platform(s), authorized third parties, etc. may be cellular, satellite, power line carrier (PLC), or radio frequency (RF) mesh. These are merely examples, and other methods may be used in other embodiments.

The data transmitted may be in the form of an alert or a critical alert. Further, the alerts may be communicated to a central computing system, operations systems, artificial intelligence platforms, authorized third parties including but not limited to utility personnel, first responders, etc.

Note that the fire mitigation and/or public safety event detection device 4700 may backhaul data, including data indicative of smoke and/or noxious gas sensors, infrared camera images, ambient and/or surface temperatures, humidity, nuclear radiation, seismic activity, voltage issues, transformer can temperatures, and weather, etc.

Regardless of where the data originates, the fire mitigation and/or public safety event detection device 4700 backhauls the data. The fire mitigation and/or public safety event detection device 4700 transmits the data to central computing devices, operations systems, artificial intelligence platforms, authorized third parties, authorized personnel including but not limited to utility personnel, first responders, etc.

Regardless of how the fire/wildfire and/or public safety event started, and the delay in responding, fires/wildfires and public safety events are a danger to human life, personal property, the environment, localized economies, wildlife, etc. Many human lives and/or injuries are at stake, priceless environmental damage is at stake, billions of dollars of property are at stake, millions or billions of dollars of lawsuit and litigation costs are at stake, and millions or billions of dollars of resulting suppression costs are at stake when a fire/wildfire and/or public safety event occurs.

In addition to fires/wildfires, a series of additional public safety events are also conceivable. For example, nuclear radiation emissions, noxious gas emissions, air quality conditions, etc. may also contribute to risk of human life, human injury, environmental damage, agricultural damage, economic impact, legal costs, suppressions costs, etc.

In addition, the fire mitigation and/or public safety event detection device 4700 may comprise an antenna (not shown). The antenna shall allow improved communications to the central computing device(s), operations systems, artificial intelligence platforms, authorized third parties, utilities, first responders, etc.

In one embodiment, the fire mitigation and/or public safety event detection device 4700 may comprise a magnet or bracket (not shown) on the back of the fire mitigation and/or public safety event detection device 4700. The magnet or bracket allow the fire mitigation and/or public safety event detection device 4700 to be attached to a power pole, a building, a transmission tower, the host transformer, the host asset(s), or other outdoor objects, e.g., tree, etc.

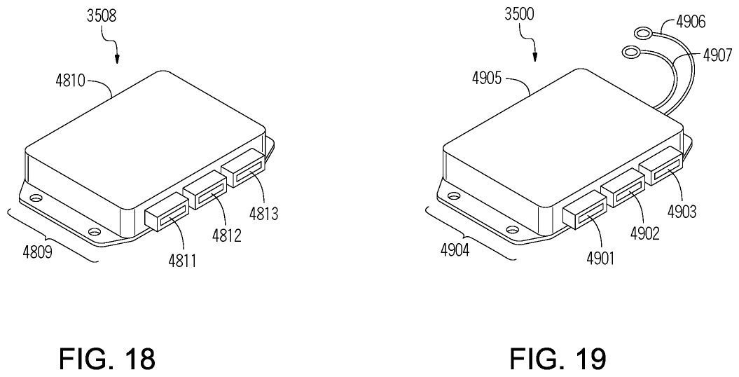

FIG. 18 is a sensor pack 3508 bearing onboard sensors. The sensor pack 3508 is substantially cuboidal although it can be of other shapes (not shown), and can be made of metal, plastic, or other conducive material. The sensor pack 3508 comprises a housing 4810 and a connector strip(s) 4809 for connecting the sensor pack 3508 to a fire mitigation and/or public safety event detection device, and/or to a separate object e.g., a tree, a building, a power pole, a transmission tower, etc. Note that there is also a connector strip on the opposing side of the external sensor pack 3508 that cannot be seen from the perspective view provided.

The sensor pack 3508 may comprise a plurality of ports such as but not limited to 4811, 4812, and 4813. Communication mediums, internal sensors and/or external sensors may be connected to the ports such as 4811, 4812, and 4813. The sensor pack 3508 of FIG. 18, however, does not comprise power source conductors. Instead, the sensor pack 3508 is battery powered by a battery within the sensor pack housing 4810 (not shown).

The sensor pack 3508 comprises a plurality of sensors. For example, the sensor pack 3508 may comprise, but are not limited to an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors.

The sensor pack 3508 may be mounted to an array of general form factor fire mitigation and/or public safety event detection devices or to other outdoor objects, e.g., power pole, tree, building, transmission tower, etc. Sensors contained in the sensor pack 3508 and/or tethered to or wirelessly connected to sensor pack 3508 detect localized surrounding environmental and/or associated host transformer or host asset(s) data and transmit the data to electronics contained within the housings of the general form factor fire mitigation and/or public safety event detection devices or to internal printed circuit boards within 3508 which include communications capabilities.

FIG. 19 is a sensor pack 3500 bearing onboard sensors. The sensor pack 3500 is substantially cuboidal although it can be of other shapes (not shown), and can be made of metal, plastic, or other conducive material. The sensor pack 3500 comprises a housing 4904 and a connector strip(s) 4904 for connecting the sensor pack 3500 to a fire mitigation and/or public safety event detection device, and/or to a separate object e.g., a tree, a building, a power pole, a transmission tower, etc. Note that there is also a connector strip on the opposing side of the external sensor pack 3500 that cannot be seen from the perspective view provided.

The sensor pack 3500 may comprise a plurality of ports such as but not limited to 4901, 4902, and 4903. Communication mediums, internal sensors and/or external sensors may be connected to the ports such as 4901, 4902, and 4903.

The sensor pack 3500 of FIG. 19 comprises power source conductors of one or more wires such as 4906 and 4907.

The sensor pack 3500 comprises a plurality of sensors. For example, the sensor pack 3500 may comprise, but are not limited to an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors.

The sensor pack 3500 may be mounted to an array of general form factor fire mitigation and/or public safety event detection devices or to other outdoor objects, e.g., power pole, tree, building, transmission tower, etc. Sensors contained in the sensor pack 3500 and/or tethered to or wirelessly connected to sensor pack 3500 detect localized surrounding environmental and/or associated host transformer or host asset(s) data, and transmit the data to electronics contained within the housings of the general form factor fire mitigation and/or public safety event detection devices, or to internal printed circuit boards within 3500 which include communications capabilities.

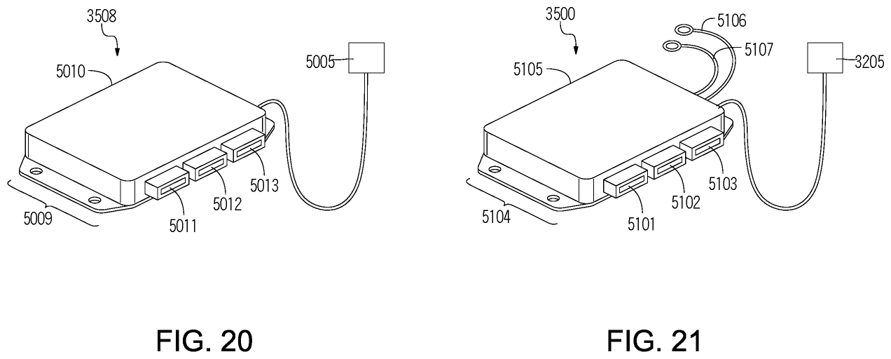

FIG. 20 is a sensor pack 3508 bearing onboard sensors. The sensor pack 3508 is substantially cuboidal although it can be of other shapes, and can be made of metal, plastic, or other conducive material. The sensor pack 3508 comprises a housing 5010 and a connector strip(s) 5009 for connecting the sensor pack 3508 to a fire mitigation and/or public safety event detection device, and/or to a separate object e.g., a tree, a building, a power pole, a transmission tower, etc. Note that there is also a connector strip on the opposing side of the external sensor pack 3508 that cannot be seen from the perspective view provided.

The sensor pack 3508 may comprise a plurality of ports such as but not limited to 5011, 5012, and 5013. Communication mediums, internal sensors and/or external sensors may be connected to the ports such as 5011, 5012, and 5013. The sensor pack 3508 of FIG. 20, however, does not comprise power source conductors. Instead, the sensor pack 3508 is battery powered by a battery within the sensor pack housing 5010 (not shown).

The sensor pack 3508 also bears a wired external sensor pack 5005.

The sensor pack 3508 and 5005 comprises a plurality of sensors. For example, the sensor pack 3508 may comprise, but are not limited to an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors.

The sensor pack 3508 may be mounted to an array of general form factor fire mitigation and/or public safety event detection devices or to other outdoor objects, e.g., power pole, tree, building, transmission tower, etc. Sensors contained in the sensor pack 3508 and/or tethered to or wirelessly connected to sensor pack 3508 such as but not limited to 5005 detect localized surrounding environmental and/or associated host transformer or asset(s) data and transmit the data to electronics contained within the housings of the general form factor fire mitigation and/or public safety event detection devices or to internal printed circuit boards which include communications capabilities.

FIG. 21 is a sensor pack 3500 bearing onboard sensors. The sensor pack 3508 is substantially cuboidal although it can be of other shapes, and can be made of metal, plastic, or other conducive material. The sensor pack 3500 comprises a housing 5105 and a connector strip(s) 5104 for connecting the sensor pack 3508 to a fire mitigation and/or public safety event detection device, and/or to a separate object e.g., a tree, a building, a power pole, a transmission tower, etc. Note that there is also a connector strip on the opposing side of the external sensor pack 3500 that cannot be seen from the perspective view provided.

The sensor pack 3500 may comprise a plurality of ports such as but not limited to 5101, 5102, and 5103. Communication mediums, internal sensors and/or external sensors may be connected to the ports such as 5101, 5102, and 5103. The sensor pack 3500 of FIG. 21 does comprise power source conductors of one or more wires such as 5106 and 5107.

The sensor pack 3500 also bears a wired external sensor 3205.

The sensor pack 3500 and wired sensor 3205 comprises a plurality of sensors. For example, the sensor packs 3500 and the wired sensor 3205 may comprise, but are not limited to an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors.

The sensor pack 3500 may be mounted to an array of general form factor fire mitigation and/or public safety event detection devices or to other outdoor objects, e.g., power pole, tree, building, transmission tower, etc. Sensors contained in the sensor pack 3500 and/or tethered to or wirelessly connected to sensor pack 3500 such as but not limited to 3205 detect localized surrounding environmental and/or associated host transformer or asset(s) data and transmit the data to electronics contained within the housings of the general form factor fire mitigation and/or public safety event detection devices or to internal printed circuit boards which include communications capabilities.

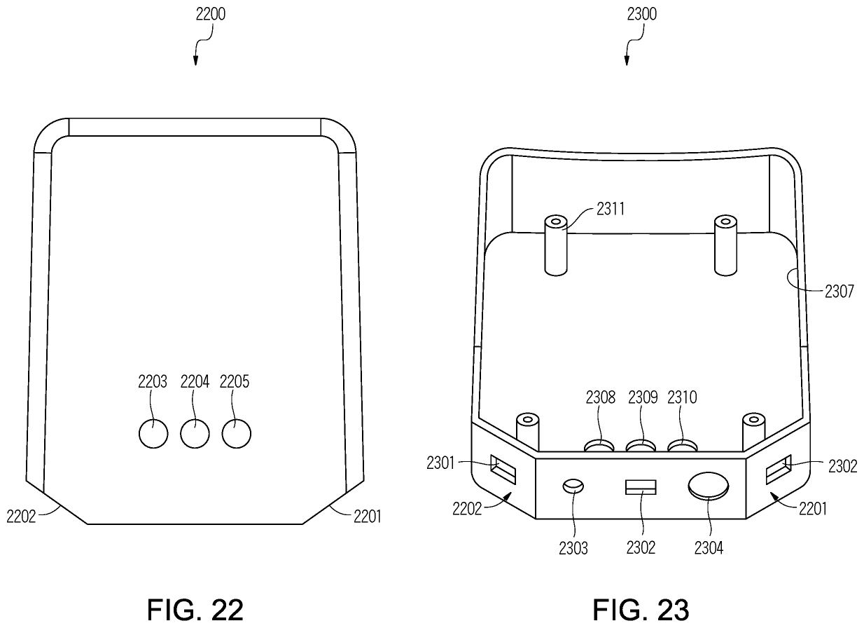

FIG. 22 is a faceplate 2200 for a fire mitigation and/or public safety event detection device. The faceplate may be made of different types of materials. For example, the faceplate 2200 may be made of metal or a durable plastic. Notably, the material out of which the faceplate is made withstands an outdoor environment.

The faceplate 2200 comprises a plurality of openings on its top. Openings 2203 and 2204 are for receiving light-emitting diodes (not shown) that may be mounted on an underlying fire mitigation and/or public safety event detection device and project through the fire mitigation and/or public safety event detection device to be exposed to the outside.

Further, the faceplate 2200 comprises another opening 2205. The opening 2205 is for receiving an optical port (not shown) that may be mounted on an underlying fire mitigation and/or public safety event detection device and project through the fire mitigation and/or public safety event detection device to be exposed to the outside.

FIG. 23 is sensor tray 2300 that comprises a substantially rectangular inner cavity 2307. The inner cavity 2307 is configured to house a plurality of sensors. The sensors may be an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors.

In one embodiment, the sensor tray 2300 houses three infrared cameras (not shown). In this regard, an infrared camera may be mounted to capture images from the substantially square opening 2304 on the face of the sensor tray 2300. Note that the sensor tray 2300 has beveled corners 2306 and 2301 creating beveled faces. An infrared camera may be mounted to capture images from the substantially square opening 2301, and an infrared camera may be mounted to capture images from the substantially square opening 2306. Using three cameras each having a different field of view provides a wider image, allowing a recipient of the images to have a larger picture of the environment.

Further, the sensor tray 2300 comprises on its front face an opening 2305. In this regard, the cavity 2307 houses a smoke sensor (not shown). The smoke senso is coupled to the opening 2305. The smoke sensor 2305 detects smoke in its environment.

Note that bottom face of the sensor tray 2300 has corresponding openings 2308, 2309, and 2310. Openings 2308 and 2309 are for receiving light-emitting diodes (not shown) that may be mounted on an underlying fire mitigation and/or public safety event detection device and project through the cavity 2307 to be exposed to the outside via the openings 2203, 3304, and 2205 (FIG. 22) in the faceplate 2200 (FIG. 22).

1. A fire mitigation and/or public safety event detection device, comprising;
a housing having a cavity;
a plurality of varying sensors coupled within the cavity of the housing;
a plurality of varying sensors externally coupled and/or wired to interface with the housing:
a processor configured to determine, based upon outputs from the sensors, whether there is a fire event or a public safety event and/or associated conditions, the processor further configured to transmit data indicative of the sensors and alerts related to the fire event or the public safety event or indicative conditions to a computing device of utility personnel, third parties, or first responders.

2. The fire mitigation and public safety event detection device of claim 1, wherein the computing device is a handheld device.

3. The fire mitigation and public safety event detection device of claim 1, wherein the computing device is a tablet.

4. The fire mitigation and public safety event detection device of claim 1, wherein the computing device is a computer.

5. The fire mitigation and public safety event detection device of claim 1, wherein the computing device is a laptop.

6. The fire mitigation and public safety event detection device of claim 1, wherein the processor is further configured to backhaul data collected from the sensors or information gleaned from the sensors to a computing device.

7. The fire mitigation and public safety event detection device of claim 6, wherein the processor is further configured to backhaul data collected from the sensors or information gleaned from the sensors to a computing device cellularly.

8. The fire mitigation and public safety event detection device of claim 6, wherein the processor is further configured to backhaul data collected from the sensors or information gleaned from the sensors to a computing device via global system for mobile communications (GSM).

9. The fire mitigation and public safety event detection device of claim 6, wherein the processor is further configured to backhaul data collected from the sensors or information gleaned from the sensors to a computing device via radio frequency mesh.

10. The fire mitigation and public safety event detection device of claim 6, wherein the processor is further configured to backhaul data collected from the sensors or information gleaned from the sensors to a computing device via a satellite.

11. The fire mitigation and public safety event detection device of claim 6, wherein the processor is further configured to backhaul data collected from the sensors or information gleaned from the sensors to a computing device via a programmable logic controller.

11. The fire mitigation and public safety event detection device of claim 6, wherein the processor is further configured to backhaul data collected from the sensors or information gleaned from the sensors to a computing device via a power line carrier.

12. The fire mitigation and public safety event detection device of claim 1, wherein one of the sensors is an infrared imaging camera that is configured and arranged to capture images in the fire mitigation and public safety event detection device environs.

13. The fire mitigation and public safety event detection device of claim 12, wherein the processor is configured to transmit an alert to the computing device if the captured images indicate fire or some other public safety scenario.

14. The fire mitigation and public safety event detection device of claim 1, wherein one of the sensors is a vibration sensor that is configured and arranged to detect abnormal vibrations.

15. The fire mitigation and public safety event detection device of claim 14, wherein the processor is configured to transmit an alert to the computing device if the output of the vibration sensor indicated abnormal vibrations.

16. The fire mitigation and public safety event detection device of claim 1, wherein one of the sensors is an ambient sensor that is configured and arranged to detect ambient temperature.

17. The fire mitigation and public safety event detection device of claim 16, wherein the processor is configured to transmit an alert to the computing device if the output of the ambient sensor indicates abnormal temperature.

18. The fire mitigation and public safety event detection device of claim 1, wherein one of the sensors is a seismic activity sensor that is configured and arranged to detect seismic activity.

19. The fire mitigation and public safety event detection device of claim 18, wherein the processor is configured to transmit an alert to the computing device if the output of the seismic activity sensor indicates abnormal seismic activity.

20. The fire mitigation and public safety event detection device of claim 1, wherein one of the sensors is an air quality sensor that is configured and arranged to detect air quality.

21. The fire mitigation and public safety event detection device of claim 20, wherein the processor is configured to transmit an alert to the computing device if the output of the air quality sensor indicates that the air quality is of concern.

22. The fire mitigation and public safety event detection device of claim 1, wherein one of the sensors is a wind direction and/or wind speed sensor that is configured and arranged to detect wind direction and speed.

23. The fire mitigation and public safety event detection device of claim 22, wherein the processor is configured to transmit an alert to the computing device if the output of the wind direction and/or speed sensor is of concern.

24. The fire mitigation and public safety event detection device of claim 1, wherein one of the sensors is a voltage sensor that is configured and arranged to detect voltage levels and/or circuit faults.

25. The fire mitigation and public safety event detection device of claim 24, wherein the processor is configured to transmit an alert to the computing device if the output of the voltage sensor indicates unexpected voltage levels, and/or circuit faults.

26. The fire mitigation and public safety event detection device of claim 1, wherein one of the sensors is a humidity sensor that is configured and arranged to detect humidity.

27. The fire mitigation and public safety event detection device of claim 26, wherein the processor is configured to transmit an alert to the computing device if the output of the humidity sensor is of concern.

28. The fire mitigation and public safety event detection device of claim 1, wherein one of the sensors is a ground and/or surface temperature sensor that is configured and arranged to detect temperature below and/or nearby a respective deployment location.

29. The fire mitigation and public safety event detection device of claim 28, wherein the processor is configured to transmit an alert to the computing device if the output of the ground and/or surface temperature sensor indicates the environment is or is not conducive to certain threats.

30. The fire mitigation and public safety event detection device of claim 1, wherein one of the sensors is a smoke sensor that is configured and arranged to detect smoke in its environs.

31. The fire mitigation and public safety event detection device of claim 30, wherein the processor is configured to transmit an alert to the computing device if the output of the smoke sensor indicates the environment is or is not conducive to certain threats.

32. The fire mitigation and public safety event detection device of claim 1, wherein one of the sensors is a nuclear radiation sensor that is configured and arranged to detect radiation in its environs.

33. The fire mitigation and public safety event detection device of claim 32, wherein the processor is configured to transmit an alert to the computing device if the output of the nuclear radiation sensor indicates there is nuclear radiation in its environs.

34. The fire mitigation and public safety event detection device of claim 1, wherein one of the sensors is a noxious gas sensor that is configured and arranged to detect noxious gas in its environs.

35. The fire mitigation and public safety event detection device of claim 26, wherein the processor is configured to transmit an alert to the computing device if the output of the noxious gas sensor indicates there is noxious gas in its environs.

36. The fire mitigation and public safety event detection device of claim 1, wherein one of the sensors is a geo-positioning sensor that is configured and arranged to indicate the latitude and longitude of its location.

37. The fire mitigation and public safety event detection device of claim 36, wherein the processor is configured to transmit an alert to the computing device if a sensor indicated a fire or public safety event and/or associated conditions, and also transmit the latitude and longitude of the sensor.

38. The fire mitigation and public safety event detection device of claim 1, wherein the housing is coupled to an external wired sensor pack containing one or more varying sensors.

39. The fire mitigation and public safety event detection device of claim 38, wherein the housing is coupled to an external power supply.

40. The fire mitigation and public safety event detection device of claim 39, wherein the housing comprising an internal battery for powering the sensors.

41. The fire mitigation and public safety event detection device of claim 40, wherein solar power is used to charge the battery.

42. The fire mitigation and public safety event detection device of claim 1, wherein a sensor pack is externally coupled or otherwise attached to the housing but communicatively coupled to electronics in the housing.

43. The fire mitigation and public safety event detection device of claim 42, wherein the sensor pack may contain an infrared imaging camera(s), vibration sensors, ambient temperature sensors, seismic activity sensors, air quality sensors, environmental wind direction and speed sensors, transformer exterior temperature sensor, humidity sensors, ground and/or surface temperature sensors detecting below and/or nearby the respective deployment location(s), smoke sensors, nuclear radiation sensors, noxious gases sensors, fault sensors, and/or geo-positioning sensors, etc.

44. The fire mitigation and public safety event detection device of claim 43, wherein the sensor pack form factor comprises a front face and two beveled faces to which any series of sensors may be affixed internally and/or externally.

45. The fire mitigation and public safety event detection device of claim 1, wherein two or more devices, each bearing one or more sensors of varying capability onboard, coupled to, and/or tethered to, may be deployed in aggregate outdoors to establish a web or network of fire mitigation and/or public safety event detection devices, thereby creating a localized monitoring, detection, and/or alerting system.

46. The fire mitigation and public safety event detection web or network of claim 45, wherein the processor is configured to transmit an alert to the computing device if the output of one or more sensors indicates that a fire/wildfire and/or public safety event, and/or associated conditions has been detected in its environs.
